(19) United States
(12) Patent Application Publication
(10) Pub. No.: US 2022/0136037 A1
(43) Pub. Date: May 5, 2022

(54) METHODS OF PREDICTING AGE, AND IDENTIFYING AND TREATING CONDITIONS ASSOCIATED WITH AGING

(71) Applicant: Seragon Pharmaceuticals, Inc., Irvine, CA (US)

(72) Inventors: Aake Vaestermark, Irvine, CA (US); George Marshall, Irvine, CA (US)

(21) Appl. No.: 17/179,976

(22) Filed: Feb. 19, 2021

Related U.S. Application Data
(60) Provisional application No. 63/110,174, filed on Nov. 5, 2020.

(51) Int. Cl. C12Q 1/6809; C12Q 1/6876

(52) U.S. Cl. CPC C12Q 1/6809; C12Q 2600/154; C12Q 1/6876

(57) ABSTRACT

Disclosed herein are methods for detecting differentially methylated CpG islands associated with epigenetic changes in a subject. Based, in part, on algorithms continually improved through machine learning, the methods and systems generate a customized report on an individual's overall health. This contains a neural network-trained assessment of the individual's genome, including differences in DNA methylation and gene expression—quantitative results which can predict the onset of developing health concerns and conditions associated with aging. The results can then be privately and conveniently accessed and shared with health providers to deliver a qualitative assessment backed by clinical judgment, and to facilitate deploying targeted treatments of identified conditions associated with aging.

### CROSS-REFERENCE TO RELATED APPLICATIONS

This application claims priority to U.S. Provisional Application No. 63/110,174, filed Nov. 5, 2020, the contents of which are incorporate by reference in their entirety.

### BACKGROUND

### Technical Field

The disclosure generally relates to the use of deep learning framework to predict the age of a subject using methylation data derived from cells, to identify conditions of a subject associated with aging. The disclosure also relates to treating conditions associated with aging, consistent with and/or based on the prediction.

### Description of the Related Art

Methylation of DNA at CpG dinucleotides is one of the most important epigenetic modifications in mammalian cells. Short regions of DNA in which the frequency of 5′-CG-3′ (CpG) dinucleotides are higher than in other regions of the genome are called CpG islands (Bird, 1986). CpG islands often harbor the promoters of genes and play a pivotal role in the control of gene expression. In normal tissue, CpG islands are usually unmethylated but a subset of islands becomes methylated during tumor development (Jones and Baylin, 2002; Costello et al., 2000; Esteller et al., 2001). Methyl-CpG binding domain (MBD) proteins specifically recognize methylated DNA sequences and are essential components of regulatory complexes that mediate transcriptional repression of methylated DNA (Hendrich and Bird, 2000; Wade, 2001). One of the best-characterized members of the MBD protein family is MBD2. MBD2 has two isoforms, MBD2a and MBD2b, which are alternatively translated from the same mRNA (Hendrich and Bird, 1998). Recent studies indicate that interacting proteins can modulate the methylated DNA-binding ability of the MBD2 protein (Jiang et al., 2004). MBD3L1 interacts with MBD2b in vivo and in vitro and promotes the formation of larger methylated-DNA binding complexes (Jiang et al., 2004).

Age prediction of humans (using methylation data) has, in the art, concentrated largely on the use of regression models. This has been done by predicting age as a single number, measured in years (PMID: 25968125). However, regression-based age predictions show only limited success when it comes to attributing complex and differential ageing to largely unknown biological systems. Difference between predicted and actual age has often been used as a strategy to address complex ageing phenotypes difficult to characterize in a rational way. In addition, attempts have been made to simultaneously and randomly alter the ageing of many genes at a time.

### SUMMARY OF THE DISCLOSURE

Provided herein are methods for detecting differences in DNA methylation in a subject. In some aspects, the method includes obtaining a DNA sample from a subject, processing the DNA sample, detecting the CpG short tandem nucleic acid sequence in the DNA sample, comparing the CpG short tandem nucleic acid sequence to a known population, and providing results to the subject. In some embodiments, the DNA sample is derived from human cells, tissues, blood, body fluids, urine, saliva, feces or a combination thereof; preferably plasma or urine free DNA. In some embodiments, comparing the CpG short tandem nucleic acid sequence in the DNA sample further comprises comparing the degree of methylation in a DNA sample from a normal population. In some embodiments, the method further includes identifying the abnormal state associated with differentially methylated CpG islands.

Also provided herein are methods for detecting a condition associated with aging. In some aspects, the method includes detecting a condition associated with aging. In some embodiments, the method includes obtaining a DNA sample from a subject, detecting the methylation of at least one of the nucleic acid sequences, or a combination thereof, comparing the DNA sample to a known population, and providing treatment to the subject based on the condition associated with aging.

The contents of this section are intended as a simplified introduction to the disclosure, and are not intended to limit the scope of any claim.

### BRIEF DESCRIPTION OF THE DRAWINGS

FIG. 1 illustrates a table of heads (top 10/353 entries) of CG lists derived from GO (Gene Ontology) terms. Terms marked with an asterisk are difficult to replace.

FIG. 2 illustrates a flow chart for approximate model design, where the key component is the number of dense layers and the size of those variable dense layers.

FIG. 3 illustrates a figure demonstrating performance of CpG_353. Y-axis is % of predictions below err. cutoff (X-axis).

FIG. 4 illustrates a figure of an in-house CpG list (blue curve) vs the Horvath CpG list (red curve) predicts the age probability distribution differently for a randomly chosen test individual aged 13.

FIG. 5 illustrates a figure for CpG islands for transport proteins (from GO), performance is better than 300 errors/10 cases.

FIG. 6 illustrates a sample profile for a 49 year old person. Based on the GenomeScore™ profile, the person's immune system, skeletal system, and receptors are 3 years older than the person's true age. On the contrary, the person's cell growth system, transporters, and adhesion proteins are 3 years younger than the chronological age. This type of intuitive diagram would likely be included in the consumer report.

FIG. 7 illustrates a figure of performance distribution from parameter scan.

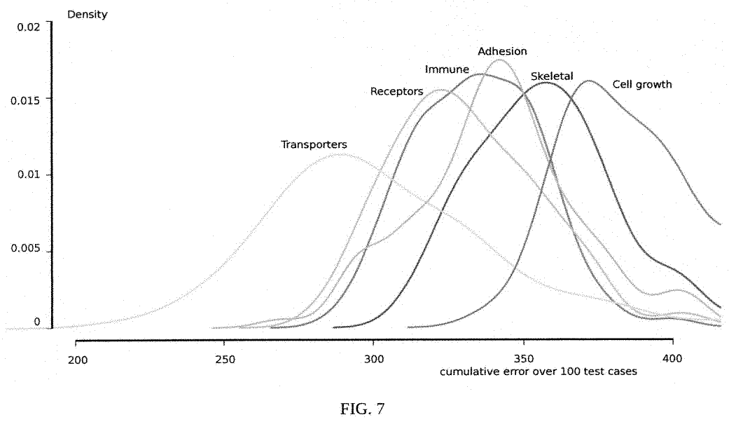

FIG. 8 illustrates a figure for CpG islands.

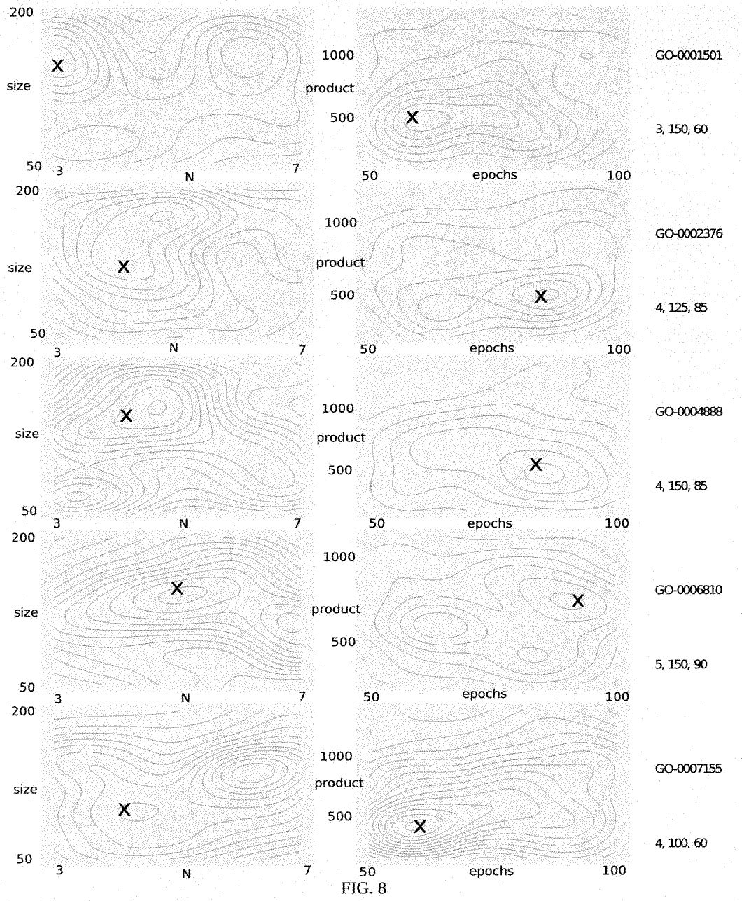

FIG. 9 illustrates an exemplary workflow of learning from data and making diagnosis.

FIG. 10 shows an exemplary computer control system that can be programmed or configured to implement the disclosed methods.

FIG. 11 shows an exemplary workflow for analyzing a biological sample.

FIG. 12A shows devices for sequencing nucleic acid samples according to one embodiment. FIG. 12B shows an analytic system that analyzes methylation status of DNA according to one embodiment.

FIG. 13 illustrates an exemplary method of feature selection from data.

FIG. 14 illustrates an exemplary method of generating diagnosis output.

FIG. 15 illustrates an example of the repeatability of prediction on non-native data in an exemplary method of age prediction.

FIG. 16 illustrates an example of co-localization of supervised prediction of condition-like data and real annotation in an exemplary method of visualization using VAE encoder transform space.

FIG. 17 shows an exemplary parameter scanning for VAE to prepare a classifier.

### DETAILED DESCRIPTION

In one aspect, the disclosure provides a method for detecting differences in DNA methylation. In some embodiments, the method comprises obtaining a DNA sample from a subject, processing the DNA sample, comparing the DNA sample to a known population, and providing results to the subject.

In some embodiments, the DNA sample is derived from human cells, tissues, blood, body fluids, urine, saliva, feces, or a combination thereof; preferably plasma or urine free DNA from humans. Free DNA in peripheral blood is mainly derived from hematopoietic cell lines; serum contains more DNA from hematopoietic cell lines than plasma free DNA, so the use of free DNA samples from plasma can reduce the background of the assay. The free DNA in the peripheral blood is excreted in the urine, so the urine also contains a large amount of free DNA. The urine test is a more non-invasive test than the peripheral blood test.

In some embodiments, the processing the DNA sample is determining the degree of methylation of the CpG short tandem nucleic acid sequence in the DNA sample. In some embodiments, the processing of the DNA sample detects more than 100 CpG islands in the subject's genome. These CpG short tandem nucleic acid sequences have three characteristics: a) a large number of copies in the human genome, and tens of thousands of CpG short tandem sequences in the human genome, so that they can be used as target sites for genome-scale detection; b) CpG short tandem sequences are highly enriched in CpG islands, so they contain important DNA methylation information that can be used to determine abnormal state of the human body; c) these CpG short tandem sequences have high melting temperatures and can therefore be effectively Amplification.

In some embodiments, comparing the DNA sample to a known population compares the degree of methylation of the CpG short tandem nucleic acid sequence in the DNA sample with the degree of methylation in a DNA sample from a normal population.

In some embodiments, providing results to the subject may comprise providing results to a physician. In some embodiments, the physician determines a subsequent health assessment.

In another aspect, a method for detecting a condition associated with aging is described herein. In some embodiments the method comprises obtaining a DNA sample from a subject; detecting the methylation of at least one of the nucleic acid sequences, or a combination thereof; comparing the DNA sample to a known population; and providing treatment to the subject based on the condition associated with aging.

In another aspect, a method for deep learning parameters to be used in detailing age prediction is provided. In some embodiments, the method comprises obtaining the following parameters: 1) size of layer; 2) number of layers; and 3) epochs.

In another aspect, the methods described herein can be applied to clinical research on the one hand, large-scale analysis to identify differentially methylated CpG islands associated with abnormal state of the human body; on the other hand, it can be applied to clinical molecular tests to predict individuals by differentially methylated CpG islands identified whether there is an abnormal state.

Another aspect of the disclosure is a deep learning-tailored version of the CpG island list presented in Genome Biol. 2013; 14(10):R115 (Supplement 3), where at least 50% of the known CpG islands have been replaced. The published list of CpG islands is known to the person skilled in the are, and is represented in Genome Biol. 2013; 14(10):R115 (Supplement 3). A “modified CpG island list” or a “synonymous CpG island list”, as used herein, means that at least 50% of the CpG islands have been replaced. In some embodiments, the predictive performance (as measured by total number of errors per 100 test cases) is maintained in the modified version of the CpG island list. In some embodiments, the modified CpG island list achieve prediction performance better than 300 errors per 10 cases, a Pearson correlation of 0.98, or a median absolute difference between predicted and chronological age of less than 3.6 years in 50% of the subjects.

Another aspect of the disclosure is a CpG island list, which encodes a biological system-specific age predictive property, according to the disclosure. In some embodiments, the list comprises system-specific CpG island sets consisting of CPG_LIST ID NO:1-5. In some embodiments, the list is biased towards a particular GO (Gene Ontology) term, displaying an FDR (False Discovery Rate) of 7.17E-07 or better. In some embodiments, an FDR rate of 6.12E-18. In some embodiments, an FDR of 4.33E-27 or better. As a non-limiting example, the modified CpG island list can be created by a “synonymy” strategy (see Methods).

Another aspect of the disclosure is the use of a noise approach to generate probability density function of age prediction, to enable system-specific comparison of a senescence process. “To enable system-specific comparison”, as used herein, means that the deep learning prediction is repeated multiple times with stochastic noise added in parallel. In some embodiments, noise taken from the distribution of a CpG island's probability of being methylated is used, when compared across systems.

Deep learning, as used herein, can be any workflow involving a tensorflow frontend, including, but not limited to the Keras package in Python. Preferably, predictions are made using one or more of the system-specific CpG island lists, see CPG_LIST ID NO:1-5. Even more preferably, the deep learning workflow parameters are selected from the 2-d parameter space ranges presented in PARAMETER_LIST ID NO:1-5.

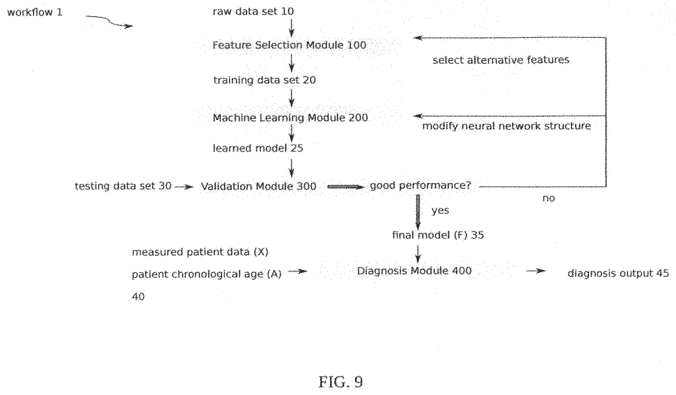

In one embodiment, an exemplary workflow 1 of learning from data and making diagnosis is shown in FIG. 9. The workflow 1 starts from obtaining a raw data set 10 and ingesting the raw data set 10 to a computer system. Raw data set 10 may comprise bioinformatic data and biological properties of a plurality of samples. Each sample may come from a specific tissue of an individual. The bioinformatic data of a sample may comprise methylation probability of CpG islands as a function of chromosomal location of the CpG islands. The biological properties of a sample may comprise the tissue type of the sample, disease state of the sample, and chronological age of the individual.

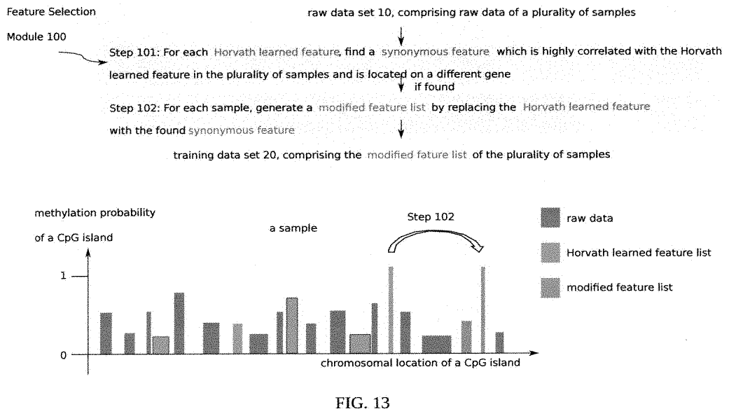

After ingesting the raw data set 10 to the computer system, the workflow 1 then inputs the raw data set 10 into a feature selection module 100 programmed in the computer system. The feature selection module 100 is configured to process the raw data set 10 and output a training data set 20. Details of the feature selection module 100 will be discussed in connection with FIG. 13.

After obtaining the training data set 20, the workflow 1 then inputs the training data set 20 to a machine learning module 200 programmed in the computer system. In some embodiments, the machine learning module 200 comprises an artificial neural network, e.g., a deep learning model provided by the Keras library. The machine learning module 200 uses the training data set 20 to inform a hypothesis which minimizes the in-sample error, and subsequently outputs a learned model 25.

After obtaining the learned model 25, the workflow 1 then inputs the learned model 25 to a validation module 300 programmed in the computer system. In addition, the workflow 1 inputs a testing data set 30 to the validation module 300. The testing data set 30 has the same kind of data as the raw data set 10, but from a different set of samples. The validation module 300 evaluates the testing data set 30 on the learned model 25, and obtains a performance, i.e., out-of-sample error.

If the performance is not good, i.e., the out-of-sample error is not smaller than a desired threshold, the workflow 1 can re-program the feature selection module 100 to select alternative features, modify the hypothesis set used in the machine learning module 200 (e.g., modify the artificial neural network structure), or both.

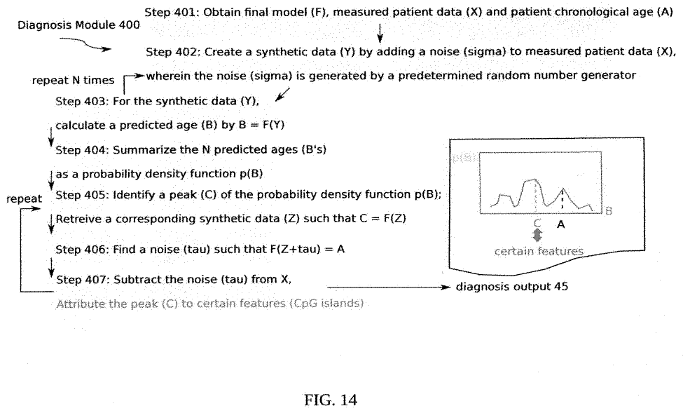

If the performance is good, i.e., the out-of-sample error is smaller than a desired threshold, the workflow 1 then outputs the learned model 25 as the final model 35. When presented with a new data 40 from a patient, the workflow 1 inputs the new data 40 and the final model 35 into a diagnosis module 400 programmed in the computer system. The diagnosis module 400 can use the new data 40 and the final model 35 to generate a diagnosis output 45 for the patient. Details of the diagnosis module 400 will be discussed in connection with FIG. 14.

### Example of a Biological Sample

A biological sample suitable for use in the methods disclosed herein can include any tissue or material derived from a living or dead subject. A biological sample can be a cell-free sample. A biological sample can comprise a nucleic acid (e.g., DNA, e.g., genomic DNA or mitochondrial DNA, or RNA) or a fragment thereof. The nucleic acid in the sample can be a cell-free nucleic acid. A sample can be a liquid sample or a solid sample (e.g., a cell or tissue sample). The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. Stool samples can also be used. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). The biological sample can be treated to physically disrupt tissue or cell structure (e.g., centrifugation and/or cell lysis), thus releasing intracellular components into a solution which can further contain enzymes, buffers, salts, detergents, and the like which are used to prepare the sample for analysis.

Methods, compositions, and systems provided herein can be used to analyze nucleic acid molecules in a biological sample. The nucleic acid molecules can be cellular nucleic acid molecules, cell-free nucleic acid molecules, or both. The cell-free nucleic acids used by methods as provided herein can be nucleic acid molecules outside of cells in a biological sample. The cell-free nucleic acid molecules can be present in various bodily fluids, e.g., blood, saliva, semen, and urine. Cell-free DNA molecules can be generated owing to cell death in various tissues that can be caused by health conditions and/or diseases, e.g., tumor invasion or growth, immunological rejection after organ transplantation.

Cell-free nucleic acid molecules, e.g., cell-free DNA, used in methods as provided herein can exist in plasma, urine, saliva, or serum. Cell-free DNA can occur naturally in the form of short fragments. Cell-free DNA fragmentation can refer to the process whereby high molecular weight DNA (such as DNA in the nucleus of a cell) are cleaved, broken, or digested to short fragments when cell-free DNA molecules are generated or released. Methods, compositions, and systems provided herein can be used to analyze cellular nucleic acid molecules in some cases, for instance, cellular DNA from a tumor tissue, or cellular DNA from white blood cells when the patient has leukemia, lymphoma, or myeloma. Sample taken from a tumor tissue can be subject to assays and analyses according to some examples of the present disclosure.

### Example of a Tissue-Specific Marker

Tissue-specific markers can identify a tissue of origin for a DNA molecule. In some cases, the tissue-specific marker is a polynucleotide sequence of the genome of an organism. In some cases, the tissue-specific marker comprises a differentiated methylated region (DMR) which is identified based on the methylation status of one or more differentiated methylation sites contained within the marker polynucleotide sequence. In some cases, the one or more differentiated methylation sites comprise one or more CpG sites. In some cases, the one or more differentiated methylation sites comprise one or more non-CpG sites. In some cases, a tissue-specific marker as discussed herein can be referred to as a target sequence.

In some cases, the differentiated methylation sites of the tissue-specific marker have a first methylation status in a first tissue of the organism, whereas a second methylation status in a different second tissue of the organism. The first and second methylation statuses can be different so that the first and second tissues can be differentiated based on the methylation status of the tissue-specific marker. In some cases, the differentiated methylation sites of the tissue-specific marker have a first methylation status in a first tissue of the organism, whereas a second methylation status in all other tissues of the organism. The first and second methylation statuses can be different so that the first tissue can be differentiated from all other tissues of the organism based on the methylation status of the tissue-specific marker.

In some cases, the tissue-specific marker comprises at least 1, at least 2, at least 3, at least 4, at least 5, at least 6, at least 7, at least 8, at least 9, at least 10, at least 12, at least 15, at least 20, at least 25, at least 30, at least 50 differentiated methylation sites. In some cases, the tissue-specific marker comprises at least 5 differentiated methylation sites. A methylated nucleotide or a methylated nucleotide base can refer to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. In this case, cytosine is not a methylated nucleotide and 5-methylcytosine is a methylated nucleotide. In another example, thymine contains a methyl moiety at position 5 of its pyrimidine ring, however, for purposes herein, thymine is not considered a methylated nucleotide when present in DNA since thymine is a typical nucleotide base of DNA. Typical nucleoside bases for DNA are thymine, adenine, cytosine and guanine. Typical bases for RNA are uracil, adenine, cytosine and guanine. Correspondingly a “methylation site” can be the location in the target gene nucleic acid region where methylation has, or has the possibility of occurring. For example a location containing CpG is a methylation site wherein the cytosine may or may not be methylated. A “site” can correspond to a single site, which can be a single base position or a group of correlated base positions, e.g., a CpG site. A methylation site can refer to a CpG site, or a non-CpG site of a DNA molecule that has the potential to be methylated. A CpG site can be a region of a DNA molecule where a cytosine nucleotide is followed by a guanine nucleotide in the linear sequence of bases along its 5′ to 3′ direction and that is susceptible to methylation either by natural occurring events in vivo or by an event instituted to chemically methylate the nucleotide in vitro. A non-CpG site can be a region that does not have a CpG dinucleotide sequence but is also is susceptible to methylation either by natural occurring events in vivo or by an event instituted to chemically methylate the nucleotide in vitro. A locus or region can correspond to a region that includes multiple sites.

The methylation status of the tissue-specific maker can comprise methylation density for individual sites within the marker region, a distribution of methylated/unmethylated sites over a contiguous region within the marker, a pattern or level of methylation for each individual methylation site within the marker that contains more than one sites, and non-CpG methylation. In some cases, the methylation status of the tissue-specific maker comprises methylation level (or methylation density) for individual differentiated methylation sites. The methylation density can refer to, for a given methylation site, a fraction of nucleic acid molecules methylated at the given methylation site over the total number of nucleic acid molecules of interest that contain such methylation site. For instance, the methylation density of a first methylation site in liver tissue can refer to a fraction of liver DNA molecules methylated at the first site over the total liver DNA molecules. In some cases, the methylation status comprises coherence of methylation/unmethylation status among individual differentiated methylation sites.

In some cases, the tissue-specific marker comprises methylation sites that are hypermethylated in a first tissue, but are hypomethylated in a second tissue. For instance, the tissue-specific marker can comprise one or more methylation sites that are hypermethylated in liver tissue, by which it can mean the one or more methylation sites have an at least 50%, at least 60%, at least 70%, at least 80%, at least 90%, or at least 95% methylation density in liver tissue; in contrast, the one or more methylation sites can have an at most 50%, at most 40%, at most 30%, at most 20%, at most 10%, at most 5%, or 0% methylation density in other tissues, such as, but not limited to, blood cells, lung, esophagus, stomach, small intestines, colon, pancreas, urinary bladder, heart, and brain. The tissue-specific marker can comprise at least 1, at least 2, at least 3, at least 4, at least 5, at least 6, at least 7, at least 8, at least 9, at least 10, at least 12, at least 15, at least 20, at least 25, at least 30, at least 50 methylation sites that are hypermethylated in a first tissue, but hypomethylated in a second tissue. In some cases, the tissue-specific marker comprises at least 5 methylation sites that are hypermethylated in a first tissue, but hypomethylated in a second tissue. The tissue-specific marker can comprise at most 300 base-pairs (bp), at most 250 bp, at most 225 bp, at most 200 bp, at most 190 bp, at most 185 bp, at most 180 bp, at most 175 bp, at most 170 bp, at most 169 bp, at most 168 bp, at most 167 bp, at most 166 bp, at most 165 bp, at most 164 bp, at most 163 bp, at most 162 bp, at most 161 bp, at most 160 bp, at most 150 bp, at most 140 bp, at most 120 bp, or at most 100 bp. In some cases, the tissue-specific marker comprises at most 166 bp.

In some cases, the tissue-specific marker comprises methylation sites that are hypomethylated in a first tissue, but are hypermethylated in a second tissue. In some cases, the tissue-specific marker comprises methylation sites that are hypomethylated in a first tissue, but are hypermethylated in other tissues. For instance, the tissue-specific marker can comprise one or more methylation sites that are hypomethylated in liver tissue, by which it can mean the one or more methylation sites have an at most 50%, at most 40%, at most 30%, at most 20%, at most 10%, at most 5%, or 0% methylation density in liver tissue; in contrast, the one or more methylation sites can have an at least 50%, at least 60%, at least 70%, at least 80%, at least 90%, or at least 95% methylation density in other tissues, such as, but not limited to, blood cells, lung, esophagus, stomach, small intestines, colon, pancreas, urinary bladder, heart, and brain. The tissue-specific marker can comprise at least 1, at least 2, at least 3, at least 4, at least 5, at least 6, at least 7, at least 8, at least 9, at least 10, at least 12, at least 15, at least 20, at least 25, at least 30, at least 50 methylation sites that are hypomethylated in a first tissue, but hypermethylated in a second tissue. In some cases, the tissue-specific marker comprises at least 5 methylation sites that are hypomethylated in a first tissue, but hypermethylated in a second tissue.

As used herein, the term “identity” or “percent identity” between two or more nucleotide or amino acid sequences can be determined by aligning the sequences for optimal comparison purposes (e.g., gaps can be introduced in the sequence of a first sequence). The nucleotides at corresponding positions can then be compared, and the percent identity between the two sequences can be a function of the number of identical positions shared by the sequences (i.e., % identity=# of identical positions/total # of positions×100). For example, a position in the first sequence may be occupied by the same nucleotide as the corresponding position in the second sequence, then the molecules are identical at that position. The percent identity between the two sequences may be a function of the number of identical positions shared by the sequences, taking into account the number of gaps, and the length of each gap, which need to be introduced for optimal alignment of the two sequences. In some cases, the length of a sequence aligned for comparison purposes is at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search can determine homology between two sequences. The homology can be between the entire lengths of two sequences or between fractions of the entire lengths of two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm can be those described in Karlin, S. and Altschul, S., Proc. Natl. Acad. Sci. USA, 90-5873-5877 (1993). Such an algorithm can be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA.

In some embodiments, methods of identifying tissue-specific markers can comprise comparing methylation status across the genome among different tissue samples. Publicly available databases, such as, databases from RoadMap Epigenomics Project (Roadmap Epigenomics Consortium et al. Nature 2015; 518:317-30) and BLUEPRINT project (Martens et al. Haematologica 2013; 98:1487-9), can be utilized for bioinformatics analysis in order to screen for potential tissue-specific markers. In some cases, experimental validation is desirable. For instance, methylation-aware sequencing, such as bisulfite sequencing, can be performed to validate the methylation status among different tissues. In some cases, methylation-specific amplification can also be used for a relatively more target-orientated validation.

As used herein, the term “differentially methylated region” or “DMR” refers to a region in chromosomal DNA that is differentially methylated (e.g., at a CpG motif) between said species of DNA and the other DNA with which it is admixed in the sample. For example in one embodiment, the DMRs used in the present invention are differentially methylated between fetal and maternal DNA, or are differentially methylated between tumor-derived and non-tumor-derived DNA from the same individual. In particular embodiments of the present invention, the DMRs are hypermethlyated in fetal DNA and hypo methylated in maternal DNA, or are hypermethylated in tumor-derived DNA and hypomethylated in DNA that is derived from non-tumor tissue of the individual. That is, in such regions exhibit a greater degree (i.e., more) methylation in said species of DNA (e.g., the fetal or tumor cfDNA) as compared to the other DNA (e.g., maternal or non-tumor DNA), such as about 10%, 20%, 30%, 40%, 50%, 60%, 70%, 80%, 90% or 100% or, or more of, the sites available for methylation at a given DMR are methylated in said species of DNA as compared to the same sites in the other DNA.

### Example of a Determination of a Methylation Pattern

In some embodiments, epigenetic modifications (cytosine methylation) on multiple methylation sites of single nucleic acid molecules are analyzed by determining the methylation status of the methylation sites covered by the methylation haplotype. As used herein, “methylation status” refers to methylation or unmethylation of a cytosine residue, for example, in a CpG dinucleotide, or in other contexts, e.g., CHG, CHH, etc. Methylated cytosine may be a variety of forms, for example, 5-methylcytosine (5mC), 5-hydroxymethylcytosine (5hmC), 5-formylcytosine (5fC) and 5-carboxycytosine (5caC), etc. A variety of protocols are available for the analysis of methylation status, with or without target enrichment (reviewed in Plongthongkum et al. 2014). For example, reduced representation bisulphite sequencing (RRBS), methylation restriction enzyme sequencing (MRE-seq), methylation DNA immunoprecipitation sequencing (MeDIP-seq, Papageorgiou et al. 2010), methyl-CpG-binding domain protein sequencing (MBD-seq), methylation DNA capture sequencing (MethylCap-seq), etc. In some embodiments, methylation status can be obtained by the standard short-read sequencing platforms, such as Illumina's HiSeq/MiSeq or LifeTech's Ion Proton, as part of the methylation assay without extra efforts and cost. In some embodiments, the methylation status may be analyzed using a target methylation sequencing technology (e.g., Bisulfite Padlock Probes, or BSPP, Deng et al, 2008; Diep et al. 2012). In some embodiments, the target nucleic acids may be enriched before the methylation status analysis. For example, micro-droplet PCR (Komori et al. 2011) or Selector probes (Johansson et al. 2011) can be used with some differences in the requirement of input materials and/or cost. Accordingly, each of the foregoing approaches may be utilized in some embodiments of the methods and compositions described herein.

In some embodiments, the determination of the methylation pattern, methylation frequency, or methylation status of the one or more candidate genes/loci may be accomplished by means of one or more methods selected from reverse-phase HPLC, thin-layer chromatography, SssI methyltransferases with incorporation of labeled methyl groups, the chloracetaldehyde reaction, differentially sensitive restriction enzymes, hydrazine or permanganate treatment (m5C is cleaved by permanganate treatment but not by hydrazine treatment), bisulfite sequencing, combined bisulfite-restriction analysis, pyrosequencing, methylation-sensitive single-strand conformation analysis (MS-SSCA), high resolution melting analysis (HRM), methylation-sensitive single nucleotide primer extension (MS-SnuPE), base-specific cleavage/MALDI-TOF, methylation-specific PCR (MSP), microarray-based methods, and MspI cleavage (reviewed, e.g., in Rein, T. et al. (1998) Nucl. Acids Res. 26: 2255-2264). Methods for detecting methylation status have been described in, for example U.S. Pat. Nos. 6,214,556; 5,786,146; 6,017,704; 6,265,171; 6,200,756; 6,251,594; 5,912,147; 6,331,393; 6,605,432; 5,786,146; 6,143,504; 6,596,493; 6,884,586; 6,300,071; and 7,195,870; and U.S. Patent Application Publication Nos. 20030148327; 20030148326; 20030143606; 20050009059; and 20060292564; each of which are incorporated herein in their entirety by reference. Alternative array based methods of methylation analysis are disclosed in U.S. Patent Application Publication No. 20050196792. See also Oakeley, E. J., (1999) Pharmacol. Ther. 84: 389-400; Fraga et al., (2002) BioTechniques 33: 632-649; and Dahl et al., (2003) Biogerontology 4: 233-250.

In some embodiments, methods for identifying methylation may be based on differential cleavage by restriction enzymes are used. Methylation-sensitive restriction analysis followed by PCR amplification or Southern analysis have been disclosed, for example, in Huang, T. H. et al. (1997) Cancer Res. 57: 1030-1034; Zuccotti, M. et al, (1993) Meth. Enzymol. 225: 557-567; Carrel, L. et al. (1996) Am. J Med. Genet. 64: 27-30; and Chang et al. (1992) Plant Mol. Biol. Rep. 10: 362-366. In some embodiments, enzymes that include at least one CpG dinucleotide in the recognition site may be used. Enzymes with a recognition site that includes the sequence CCGG include, for example, MspI, HpaII, AgeI, XmaI, SmaI, NgoMIV, Noel, and BspEI. Enzymes with a recognition site that includes the sequence CGCG include, for example, BstUI (CGCG, MSRE), MluI (ACGCGT, MSRE), SacII (CCGCGG, MSRE), BssHII (GCGCGC, MSRE) and NruI (TCGCGA, MSRE). NotI, BstZI, CspI and EagI have two CpGs in their recognition sites and cleavage is blocked by CpG methylation. Enzymes with a recognition site that includes the sequence GCGC include, for example, HinPlI, HhaI, AfeI, KasI, NarI, SfoI, BbeI, and FspI. Enzymes with a recognition site that includes the sequence TCGA include, for example, TaqI, ClaI (MSRE), BspDI (MSRE), PaeR7I, TliI, XhoI, SalI, and BstBI. For additional enzymes that contain CpG in the recognition sequence and for information about the enzyme's sensitivity to methylation, see, for example, the New England Biolabs catalog and web site. In some aspects two restriction enzymes may have a different recognition sequence but generate identical overhangs or compatible cohesive ends. For example, the overhangs generated by cleavage with HpaII or MspI can be ligated to the overhang generated by cleavage with TaqI. Some restriction enzymes that include CpG in the recognition site are unable to cleave if the site is methylated; these are methylation sensitive restriction enzymes (MSRE). Other enzymes that contain CpG in their recognition site can cleave regardless of the presence of methylation; these are methylation insensitive restriction enzymes (MIRE). A third type of enzyme cleaves only when the recognition site is methylated, and are referred to herein as methylation dependent restriction enzymes (MDRE). Examples of MIREs that have a CpG in the recognition sequence include, for example, BsaWI (WCCGGW), BsoBI, BssSI, MspI, and TaqI. Examples of MSREs, that include a CpG in the recognition site, include AatII, AciI, AclI, AfeI, AgeI, AscI, AvaI, BmgBI, BsaAI, BsaHI, BspDI, ClaI, EagI, FseI, PauI, HaeIII, HpaII, HinPII, MluI, NarI, NotI, NruI, PvuI, SacII, SalI, SmaI and SnaBI. In preferred aspects a pair of enzymes that have differential sensitivity to methylation and cleave at the same recognition sequence with one member of the pair being a MSRE and the other member being MIRE is used. Still other enzymes include BthCI, GlaI, HpaI, HinPlI, DpnI, MboI, ChaI and BstKTI.

In some embodiments, use of digital PCR technique can allow the direct determination of the actual number of the target DNA molecules without the need of calibrators. Other technologies, such as certain sequencing-based methods, such as, but not limited to, bisulfite sequencing and non-bisulfite-based methylation-aware sequencing using the PacBio sequencing platform, can determine the relative or fractional concentration of the DNA from the target tissues in relation to other tissues. The absolute amount can refer to an absolute count of DNA molecules, or in some cases, can also refer to a concentration of DNA molecules, e.g., number, mole, or weight per volume, e.g., copies/mL, mole/L, or mg/L. The analysis of the absolute amount as provided herein can be useful in scenarios when increased amounts of DNA would be released from more than one type of tissues. Methylation deconvolution analysis, based on sequencing of cell-free nucleic acid molecules, such as disclosed in U.S. patent application Ser. No. 14/803,692, on the other hand, can provide readout of tissue of origin of cell-free nucleic acids in the form of fractional contribution, e.g., a first tissue contributes A % of cell-free nucleic acids from a biological sample, and a second tissue contributes B % of cell-free nucleic acids from the same biological sample.

In some embodiments, technologies like real-time PCR, sequencing and microarray for methylation analysis of cell-free nucleic acids may be used. In some cases, the absolute number of cell-free nucleic acids harboring a tissue-specific marker, such as counting positive reactions in a digital PCR assay, may not be derived directly from methylation analysis by some technologies. However, such absolute number can be calculated indirectly based on concentrations (relative or fractional) of cell-free nucleic acids harboring tissue-specific markers, for instance, by taking the total number or concentration of cell-free nucleic acids in a given volume of biological sample into account. In some cases, the sequencing that can be used in the methods provided herein can include chain termination sequencing, hybridization sequencing, Illumina sequencing (e.g., using reversible terminator dyes), ion torrent semiconductor sequencing, mass spectrophotometry sequencing, massively parallel signature sequencing (MPSS), Maxam-Gilbert sequencing, nanopore sequencing, polony sequencing, pyrosequencing, shotgun sequencing, single molecule real time (SMRT) sequencing, SOLiD sequencing (hybridization using four fluorescently labeled di-base probes), universal sequencing, or any combination thereof. Microarrays having probes targeting methylation sites can also be used for analyzing methylation status of the cell-free DNA molecules in the methods provided herein.

### Example of Determination of Methylation Pattern Using Bisulfite Sequencing

In some embodiments, bisulfite sequencing is used for generating methylation data at single-base resolution. The term “bisulfite conversion” refers to a biochemical process for converting unmethylated cytosine residue to uracil or thymine residues, whereby methylated cytosine residues are preserved. “Bisulfite conversion” may be carried out computationally from a nucleic acid sequence contained in a computer file (such as those in FASTA, FASTQ or any file format known in the art), wherein all cytosine residues in a sequence of interest are changed to thymine or uracil residues. Exemplary reagents for bisulfite conversion include sodium bisulfite and magnesium bisulfite. “Bisulfite reagent” refers to a reagent comprising bisulfite, disulfite, hydrogen sulfite or combinations thereof, useful as disclosed herein to distinguish between methylated and unmethylated CpG dinucleotide sequences. One way to obtain such methylation data is to sequence the entire epigenome directly. Due to the difficulty in mapping bisulfite converted sequence reads and the methylation heterogeneity in a cell population, approximately 100 gigabases (Gb) of sequence data would be needed to generate a high-resolution human DNA methylation map (Lister, R. et al., (2009) Nature, 462(7271): 315-322). Other methylation profiling approaches include array capture (Hodges, E. et al., (2009) Genome Res. 19(9): 1593-1605), padlock probe capture (Deng, J. et al., (2009) Nat. Biotech. 27: 353-360; Ball, M. P. et al., (2009) Nat, Biotech., 27(4): 361-368) and reduced representation bisulfite sequencing (Gu et al., (2010) Nat. Methods 7(2): 133-136).

In particular, bisulfite sequencing involves conversion of unmethylated cytosine to uracil or thymine through a three-step process during sodium bisulfite modification. The steps are sulfonation to convert cytosine to cytosine sulfonate, deamination to convert cytosine sulfonate to uracil sulfonate or thymine sulfonate and alkali desulfonation to convert uracil sulfonate to uracil or thymine sulfonate to thymine. Conversion of methylated cytosine is much slower and is not observed at significant levels in a 4-16 hour reaction (Clark, S. J. et al, (1994) Nucleic Acids Res., 22(15): 2990-7). If the cytosine is methylated it will remain a cytosine. If the cytosine is unmethylated, it will be converted to uracil or thymine. When the modified strand is copied, for example, extension of a locus specific primer, a random or degenerate primer or a primer to an adaptor, a G will be incorporated in the interrogation position (opposite the C being interrogated) if the C was methylated and an A will be incorporated in the interrogation position if the C was unmethylated. When the double stranded extension product is amplified, those Cs that were converted to Us or Ts and resulted in incorporation of A in the extended primer will be replaced by Ts during amplification. Those Cs that were not modified and resulted in the incorporation of G will remain as C. Bisulfite treatment can degrade the DNA making it difficult to amplify. The sequence degeneracy resulting from the treatment also complicates primer design. The treatment may also result in incomplete desulfonation, depurination and other as yet uncharacterized DNA damage, making downstream processing more challenging. The treatment can also result in preferential amplification of unmethylated DNA relative to methylated DNA. This may be mitigated by increasing the PCR extension time.

Kits for DNA bisulfite modification are commercially available from, for example, Human Genetic Signatures' Methyleasy and Chemicon's CpGenome Modification Kit. See also, WO04096825, which describes bisulfite modification methods and Olek, A. et al. (1994) Nucl. Acids Res. 24(24): 5064-6, which discloses methods of performing bisulfite treatment and subsequent amplification on material embedded in agarose beads. In some aspects a catalyst such as diethylenetriamine may be used in conjunction with bisulfite treatment, see Komiyama, M. and Oshima, S., (1994) Tetrahedron Lett. 35(44): 8185-8188. Diethylenetriamine has been shown to catalyze bisulfite ion-induced deamination of 2′-deoxycytidine to 2′-deoxyuridine at pH 5 efficiently. Other catalysts include ammonia, ethylene-diamine, 3,3′-diaminodipropylamine, and spermine. In some aspects, deamination is performed using sodium bisulfite solutions of 3-5 M with an incubation period of 12-16 hours at about 50° C. A faster procedure has also been reported using 9-10 M bisulfite pH 5.4 for about 10 minutes at 90° C., see Hayatsu, H. et al., (2004) Proc. Jpn. Acad. Ser. B 80(4): 189-194.

Bisulfite treatment allows the methylation status of cytosines to be detected by a variety of methods. For example, any method that may be used to detect a single nucleotide polymorphism (SNP) may be used, for examples, see Syvanen, A. C. (2001) Nature Rev. Gen. 2(12): 930-942. In a preferred aspect, bisulfite sequencing methods, systems, and computer program products described herein may provide information regarding not only methylation frequencies or methylation status of a sequence of interest at single base resolution, but also information regarding SNPs, preferably in the same sequencing run. Other methods such as single base extension (SBE) may be used or hybridization of sequence specific probes similar to allele specific hybridization methods. “Variants” or “alleles” generally refer to one of a plurality of species each encoding a similar sequence composition, but with a degree of distinction from each other. The distinction may include any type of variation known to those of ordinary, skill in the related art, that include, but are not limited to, polymorphisms such as SNPs, insertions or deletions (the combination of insertion/deletion events are also referred to as “indels”), differences in the number of repeated sequences (also referred to as tandem repeats), and structural variations. Detection of such variants or alleles is also within the ambit of the subject matter described herein.

In some embodiments, the disclosed methods use commercial sequencing by synthesis platforms, such as the Genome Sequencer from Roche/454 Life Sciences, the Genome Analyzer from Illumina/Solexa, the SOLiD system from Applied BioSystems, Pacific Biosystems and the Heliscope system from Helicos Biosciences. Exemplary sequencing platforms may have one or more of the following features: 1) four differently optically labeled nucleotides are utilized (e.g., Genome Analyzer); 2) sequencing-by-ligation is utilized (e.g., SOLiD); 3) pyrosequencing is utilized (e.g., Roche/454); and 4) four identically optically labeled nucleotides are utilized (e.g., Helicos).

Such sequencing reactions and assays include sequencing by ligation methods commercialized by Applied Biosystems (e.g., SOLiD sequencing). In general, double stranded fragment nucleic acid molecules can be prepared by the methods described herein, and then incorporated into a water-in-oil emulsion along with polystyrene beads and amplified, for example by PCR. In some cases, alternative amplification methods can be employed in the water-in-oil emulsion such as any of the methods provided herein. The amplified product in each water microdroplet formed by the emulsion can interact, bind, or hybridize with the one or more beads present in that microdroplet leading to beads with a plurality of amplified products of substantially one sequence. When the emulsion is broken, the beads float to the top of the sample and are placed onto an array. The methods can include a step of rendering the nucleic acid bound to the beads single-stranded or partially single stranded. Sequencing primers are then added along with a mixture of four different fluorescently labeled oligonucleotide probes. The probes bind specifically to the two bases in the nucleic acid molecule to be sequenced immediately adjacent and 3′ of the sequencing primer to determine which of the four bases are at those positions. After washing and reading the fluorescence signal from the first incorporated probe, a ligase is added. The ligase cleaves the oligonucleotide probe between the fifth and sixth bases, removing the fluorescent dye from the nucleic acid molecule to be sequenced. The whole process is repeated using a different sequence primer until all of the intervening positions in the sequence are imaged. The process allows the simultaneous reading of millions of DNA fragments in a “massively parallel” manner. This “sequence-by-ligation” technique uses probes that encode for two bases rather than just one, allowing error recognition by signal mismatching and leading to increased base determination accuracy.

Alternative sequencing methods include sequencing by synthesis methods commercialized by 454/Roche Life Sciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; and 7,323,305. In general, double stranded fragment nucleic acid molecules can be prepared by the methods described herein, immobilized onto beads, and compartmentalized in a water-in-oil PCR emulsion. In some cases, alternative amplification methods can be employed in the water-in-oil emulsion such as any of the methods provided herein. When the emulsion is broken, amplified fragments remain bound to the beads. The methods can include a step of rendering the nucleic acid bound to the beads single stranded or partially single stranded. The beads can be enriched and loaded into wells of a fiber optic slide so that there is approximately 1 bead in each well. Nucleotides are flowed across and into the wells in a fixed order in the presence of polymerase, sulfhydrolase, and luciferase. Addition of nucleotides complementary to the target strand can result in a chemiluminescent signal that is recorded, such as by a camera. The combination of signal intensity and positional information generated across the plate allows software to determine the DNA sequence.

Other sequencing methods include those commercialized by Hclicos BioSciences Corporation (Cambridge, Mass.) as described in U.S. patent application Ser. No. 11/167,046, and U.S. Pat. Nos. 7,501,245; 7,491,498; 7,276,720; and in U.S. Patent Application Publication Nos. 20090061439; 20080087826; 20060286566; 20060024711; 20060024678; 20080213770; and 20080103058. In general, double stranded fragment nucleic acid molecules can be isolated and purified, then immobilized onto a flow-cell surface. The methods can include a step of rendering the nucleic acid bound to the flow-cell surface stranded or partially single stranded. Polymerase and labeled nucleotides are then flowed over the immobilized DNA. After fluorescently labeled nucleotides are incorporated into the DNA strands by a DNA polymerase, the surface is illuminated with a laser, and an image is captured and processed to record single molecule incorporation events to produce sequence data.

Other methods include sequencing by ligation methods commercialized by Dover Systems. Generally, oligonucleotides, primers, and probes can be prepared by the methods described herein. The nucleic acid molecules can then be amplified in an emulsion in the presence of magnetic beads. Any amplification methods can be employed in the water-in-oil emulsion. The resulting beads with immobilized clonal nucleic acid polonies are then purified by magnetic separation, capped, amine functionalized, and covalently immobilized in a series of flow cells. The methods can include a step of rendering the nucleic acid bound to the flow-cell surface stranded or partially single stranded. A series of anchor primers are flowed through the cell, where they hybridize to the synthetic oligonucleotide sequences at the 3′ or 5′ end of proximal or distal genomic DNA tags. Once an anchor primer is hybridized, a mixture of fully degenerate nonanucleotides (“nonamers”) and T4 DNA ligase is flowed into the cell. Each of the nonamer mixture's four components is labeled with one of four fluorophores, which correspond to the base type at the query position. The fluorophore-tagged nonamers selectively ligate onto the anchor primer, providing a fluorescent signal that identifies the corresponding base on the genomic DNA tag. Once the probes are ligated, fluorescently labeling the beads, the array is imaged in four colors. Each bead on the array will fluoresce in only one of the four images, indicating whether there is an A, C, G, or T at the position being queried. After imaging, the array of annealed primer-fluorescent probe complex, as well as residual enzyme, are chemically striped using guanidine HCl and sodium hydroxide. After each cycle of base reads at a given position have been completed, and the primer-fluorescent probe complex has been stripped, the anchor primer is replaced, and a new mixture of fluorescently tagged nonamers is introduced, for which the query position is shifted one base further into the genomic DNA tag. Seven bases are queried in this fashion, with the sequence performed from the 5′ end of the proximal tag, followed by six base reads with a different anchor primer from the 3′ end of the proximal tag, for a total of 13 base pair reads for this tag. This sequence is then repeated for the 5′ and 3′ ends of the distal tag, resulting in another 13 base pair reads. The ultimate result is a read length of 26 bases (thirteen from each of the paired tags). However, it is understood that this method is not limited to 26 base read lengths.

Other methods for sequencing include those commercialized by Illumina as described U.S. Pat. Nos. 5,750,341; 6,306,597; and 5,969,119. In general, oligonucleotides, primers, and probes can be prepared by the methods described herein to produce amplified nucleic acid sequences tagged at one (e.g., (A)/(A′) or both ends (e.g., (A)/(A′) and (C)/(C′)). In some cases, single stranded nucleic acid tagged at one or both ends is amplified by the methods described herein (e.g., by SPIA or linear PCR). The resulting nucleic acid is then denatured and the single stranded amplified nucleic acid molecules are randomly attached to the inside surface of flow-cell channels. Unlabeled nucleotides are added to initiate solid-phase bridge amplification to produce dense clusters of double-stranded DNA. To initiate the first base sequencing cycle, four labeled reversible terminators, primers, and DNA polymerase are added. After laser excitation, fluorescence from each cluster on the flow cell is imaged. The identity of the first base for each cluster is then recorded. Cycles of sequencing are performed to determine the fragment sequence one base at a time. For paired-end sequencing, such as for example, when the nucleic acid molecules are labeled at both ends by the methods described herein, sequencing templates can be regenerated in-situ so that the opposite end of the fragment can also be sequenced.

Still other sequencing methods include those commercialized by Pacific Biosciences as described in U.S. Pat. Nos. 7,462,452; 7,476,504; 7,405,281; 7,170,050; 7,462,468; 7,476,503; 7,315,019; 7,302,146; 7,313,308; and U.S. Patent Application Publication Nos. US20090029385; US20090068655; US20090024331; and US20080206764. In general, oligonucleotides, primers and probes can be prepared by the methods described herein. Target nucleic acid molecules can then be immobilized in zero mode waveguide arrays. The methods may include a step of rendering the nucleic acid bound to the waveguide arrays single stranded or partially single stranded. Polymerase and labeled nucleotides are added in a reaction mixture, and nucleotide incorporations are visualized via fluorescent labels attached to the terminal phosphate groups of the nucleotides. The fluorescent labels are clipped off as part of the nucleotide incorporation. In some cases, circular templates are utilized to enable multiple reads on a single molecule.

Another example of a sequencing technique that can be used in the methods described herein is nanopore sequencing (see e.g. Soni, G. V. and Meller, A. (2007) Clin. Chem. 53: 1996-2001). A nanopore can be a small hole of the order of one nanometer in diameter. Immersion of a nanopore in a conducting fluid and application of a potential across it can result in a slight electrical current due to conduction of ions through the nanopore. The amount of current that flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore can represent a reading of the DNA sequence.

Yet another example of a sequencing technique that can be used is semiconductor sequencing provided by Ion Torrent (e.g., using the Ion Personal Genome Machine (PGM)). Ion Torrent technology can use a semiconductor chip with multiple layers, e.g., a layer with micro-machined wells, an ion-sensitive layer, and an ion sensor layer. Nucleic acids can be introduced into the wells, e.g., a clonal population of single nucleic can be attached to a single bead, and the bead can be introduced into a well. To initiate sequencing of the nucleic acids on the beads, one type of deoxyribonucleotide (e.g., dATP, dCTP, dGTP, or dTTP) can be introduced into the wells. When one or more nucleotides are incorporated by DNA polymerase, protons (hydrogen ions) are released in the well, which can be detected by the ion sensor. The semiconductor chip can then be washed and the process can be repeated with a different deoxyribonucleotide. A plurality of nucleic acids can be sequenced in the wells of a semiconductor chip. The semiconductor chip can comprise chemical-sensitive field effect transistor (chemFET) arrays to sequence DNA (for example, as described in U.S. Patent Application Publication No. 20090026082). Incorporation of one or more triphosphates into a new nucleic acid strand at the 3′ end of the sequencing primer can be detected by a change in current by a chemFET. An array can have multiple chemFET sensors.

In some embodiments, the sequence reads may be aligned to a reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene.

From the sequence reads, the location and methylation state for each of CpG site may be determined based on alignment to a reference genome. Further, a methylation state vector for each fragment may be generated specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (e.g., denoted as I). The methylation state vectors may be stored in temporary or persistent computer memory for later use and processing. Further, duplicate reads or duplicate methylation state vectors from a single subject may be removed. In an additional embodiment, it may be determined that a certain fragment has one or more CpG sites that have an indeterminate methylation status. Such fragments may be excluded from later processing or selectively included where downstream data model accounts for such indeterminate methylation statuses.

### Targeted Bisulfite Sequencing Using Padlock Probes

In some embodiments, molecular inversion probes (MIP), described in Hardenbol, P. et al. Genome Res. 15:269-275 (2005) and in U.S. Pat. No. 6,858,412, may be used to determine methylation status after methylation dependent modification. A MIP may be designed for each cytosine to be interrogated. In a preferred aspect the MIP includes a locus specific region that hybridizes upstream and one that hybridizes downstream of an interrogation site and can be extended through the interrogation site, incorporating a base that is complementary to the interrogation position. The interrogation position may be the cytosine of interest after bisulfite modification and amplification of the region and the detection can be similar to detection of a polymorphism. Separate reactions may be performed for each NTP so extension only takes place in the reaction containing the base corresponding to the interrogation base or the different products may be differentially labeled.

Padlock Probe (PP) technology is a multiplex genomic enrichment method allowing for accurate targeted high-throughput sequencing. PP technology has been used to perform highly multiplexed genotyping, digital allele quantification, targeted bisulfate sequencing, and exome sequencing. Padlock probe technology may utilize a linear oligonucleotide molecule with two binding sequences at each end joined by a common linker sequence. The probe's binding arms may be hybridized several base pairs apart surrounding a target single-stranded genomic DNA region. A DNA polymerase (with each of the four standard dNTP molecules) may be used to fill in the gap between the two binding arms, and a DNA ligase may be used to circularize the resulting molecule. A mixture of exonucleases may then used to digest all arm; the resulting circular molecules can be amplified using rolling circle amplification or polymerase chain reaction to generate a DNA library compatible with modern high-throughput sequencers. Padlock probes are generally designed to have binding arms with complementary sequence around a chosen target region of approximately 100-200 base pairs; each binding arm is generally designed to have a specific DNA melting temperature.

The term “padlock probe” (PLP) refers to circularized nucleic acid molecules which may combine specific molecular recognition and universal amplification (or specific amplification and general recognition), thereby increasing sensitivity and multiplexing capabilities without limiting the range of potential target organisms. PLPs are long oligonucleotides of approximately 100 bases (but can be of any length), containing target complementary regions (referred to herein as “target-capturing sequences”) at both their 5′ and 3′ ends. These regions recognize adjacent sequences on the target nucleic acid sequence (Nilsson, M., et al. (1994) Science 265: 2085-2088) and may also contain “binding arms” which comprise “extension arms” having priming sites (e.g., universal priming sites”), sites recognized by ligase enzymes, and unique sequence identifiers, sometimes referred to as a “ZipCode” or “barcode”. Upon hybridization, the ends of the probes are situated into adjacent position, and can be joined by enzymatic ligation at the ligation sites (also referred to herein as “ligation arms”) converting the probe into a circular molecule (also known in the art and referred to herein as an “amplicon”) that is threaded on the target strand. This ligation and the resulting circular molecule can only take place when both ligation arm and extension arm segments recognize their target sequences correctly. Non-circularized probes may be removed by exonuclease treatment, while the circularized entities may be amplified with universal primers, which may or may not contain barcode or ZipCode sequences. This mechanism ensures reaction specificity, even in a complex nucleotide extract with a large number of padlock probes. Subsequently, the target-specific products are detected by a universal cZipCode microarray (Shoemaker, D. D., et al., (1996) Nat. Genet. 14: 450-456). PLPs have high specificity and multiplexing capabilities in gcnotyping assays (Hardcnbol, P., et al., (2003) Nat. Biotechnol., 21: 673-678.).

In some embodiments, the disclosed methods characterize the DNA methylation profile of a sample using bisulfite sequencing and include mapping a genomic sequence of interest to determine methylation status, methylation frequency, and detection of single nucleotide polymorphisms. During bisulfite sequencing, all cytosines present in the DNA molecule except those that are methylated are converted to thymines. Almost every methylated cytosine is present as a cytosine-guanine dinucleotide (CpG), though not all CpGs are methylated. In such embodiments, after the genome/chromosome is loaded into memory, the software application or computer program product performs an in silico bisulfite conversion, computationally converting all cytosines except those present in a CpG to thymines. The application or program then designs probes as previously, but penalizes the inclusion of CpG sites in each binding arm. During linker sequence insertion, the software application or computer program product generates multiple probes for those arm pairs containing a CpG; one probe assumes a methylated state (and contains a CpG dinucleotide) while the other assumes an unmethylated state (and contains a TpG instead). This procedure allows for efficient targeted bisulfite sequencing of hundreds of thousands of CpG sites in parallel.

In some embodiments, the disclosed methods use the probes or primers to obtain sequence reads of a target genome or sequence of interest by bisulfite sequencing and loading it into memory. A software application or computer program product encodes the sequence reads by predicting the forward and reverse orientation of each of the sequence reads to generate at least one forward sequence read and at least one reverse sequence read. The forward and reverse sequence reads are then converted by the software application or computer program product by computationally changing all cytosine residues in the forward sequence reads to thymine residues in silica, and changing all guanine residues to adenine residues in the reverse sequence reads. The bisulfite-converted genome sequence and all forward and reverse sequence reads are then aligned computationally by an alignment software application or computer program (e.g., ELAND, SOAP2Align, Bowtie, BWA, BLAST or any other alignment program known in the art). The alignment application or program can be a stand-alone application or integrated into the system, software application or computer program product described herein. The aligned sequences are then combined to create a map of the target genomic sequence. The software application or computer program product then analyzes and computes methylation frequencies or methylation status of the mapped sequences in entirety. In preferred embodiments, the mapped sequences may also be analyzed by the software application or computer program product for the presence of single nucleotide polymorphisms. Because bisulfite sequencing provides sequence read information at single-base resolution, this technique (and modifications thereof described in the methods, systems, and computer program products described herein) is particularly advantageous for calculating methylation frequencies and detecting SNPs in a single sequencing reaction.

In one example, to specifically capture an arbitrary subset of genomic targets for single-molecule bisulfite sequencing and digital quantification of DNA methylation at single-nucleotide resolution, the disclosed method may include: A set of 30,000 padlock probes was designed to assess the methylation state of 66,000 CpG sites within 2,020 CpG islands on human chromosome 12, chromosome 20, and 34 selected regions. To investigate epigenetic differences associated with de-differentiation, methylation in three human fibroblast lines and eight human pluripotent stem cell lines was compared. Chromosome-wide methylation patterns were similar among all lines studied, but cytosine methylation was slightly more prevalent in the pluripotent cells than in the fibroblasts. Induced pluripotent stem (iPS) cells appeared to display more methylation than embryonic stem cells. In fibroblasts and pluripotent cells, 288 regions were methylated differently. This targeted approach is particularly useful for analyzing DNA methylation in large genomes. Padlock probes have been previously used for exon capture and resequencing (Porreca, G. J. et al. (2007) Nat. Methods 4: 931-936). This approach to targeted bisulfite sequencing involves the in situ synthesis of long (150 nt) oligonucleotides on programmable microarrays, followed by their cleavage and enzymatic conversion into padlock probes. A library of padlock probes was annealed to the template DNA, circularized, and amplified by PCR before shotgun sequencing. There are, however, two major challenges in performing padlock capture for bisulfite sequencing. First, bisulfite treatment converts all unmethylated cytosines into uracils, resulting in marked reduction of sequence complexity. Achieving specific target capture on bisulfite-converted DNA is more difficult than on native genomic DNA. Second, low capturing sensitivity, high bias and random losses of alleles was initially observed with the “eMIP method” previously disclosed in the art (Porreca, G. J. et al. (2007) Nat. Methods 4: 931-936). Obtaining accurate and efficient quantification of DNA methylation was not possible with the existing protocol, especially with the presence of allelic drop-outs.

An exemplary workflow of targeted bisulfite sequencing using padlock probes includes the steps of: padlock probe design, padlock probe production, multiplex capture on bisulfate-converted DNA, capture circles amplification, shotgun sequencing library construction, followed by read mapping and data analysis.

### Examples of Computer Systems

Any of the methods disclosed herein can be performed and/or controlled by one or more computer systems. In some examples, any step of the methods disclosed herein can be wholly, individually, or sequentially performed and/or controlled by one or more computer systems. Any of the computer systems mentioned herein can utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems can be interconnected via a system bus. Additional subsystems include a printer, keyboard, storage device(s), and monitor that is coupled to display adapter. Peripherals and input/output (I/O) devices, which couple to I/O controller, can be connected to the computer system by any number of connections known in the art such as an input/output (I/O) port (e.g., USB, FireWire®). For example, an I/O port or external interface (e.g., Ethernet, Wi-Fi, etc.) can be used to connect computer system to a wide area network such as the Internet, a mouse input device, or a scanner. The interconnection via system bus allows the central processor to communicate with each subsystem and to control the execution of a plurality of instructions from system memory or the storage device(s) (e.g., a fixed disk, such as a hard drive, or optical disk), as well as the exchange of information between subsystems. The system memory and/or the storage device(s) can embody a computer readable medium. Another subsystem is a data collection device, such as a camera, microphone, accelerometer, and the like. Any of the data mentioned herein can be output from one component to another component and can be output to the user.

A computer system can include a plurality of the same components or subsystems, e.g., connected together by external interface or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

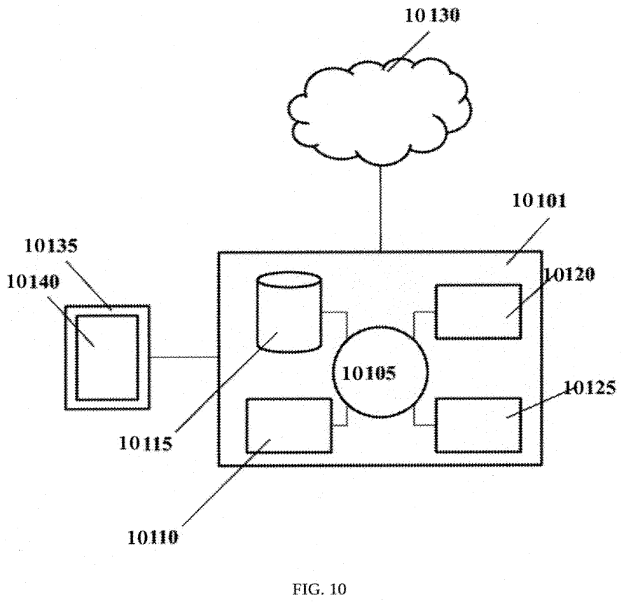

The present disclosure provides computer control systems that are programmed to implement methods of the disclosure. FIG. 10 shows a computer system 10101 that is programmed or otherwise configured to determine an absolute amount of cell-free nucleic acid molecules from a tissue of an organism as described herein. The computer system 10101 can implement and/or regulate various aspects of the methods provided in the present disclosure, such as, for example, controlling sequencing of the nucleic acid molecules from a biological sample, performing various steps of the bioinformatics analyses of sequencing data as described herein, integrating data collection, analysis and result reporting, and data management. The computer system 10101 can be an electronic device of a user or a computer system that is remotely located with respect to the electronic device. The electronic device can be a mobile electronic device.

The computer system 10101 includes a central processing unit (CPU, also “processor” and “computer processor” herein) 10105, which can be a single core or multi core processor, or a plurality of processors for parallel processing. The computer system 10101 also includes memory or memory location 10110 (e.g., random-access memory, read-only memory, flash memory), electronic storage unit 10115 (e.g., hard disk), communication interface 10120 (e.g., network adapter) for communicating with one or more other systems, and peripheral devices 10125, such as cache, other memory, data storage and/or electronic display adapters. The memory 10110, storage unit 10115, interface 10120 and peripheral devices 10125 are in communication with the CPU 10105 through a communication bus (solid lines), such as a motherboard. The storage unit 10115 can be a data storage unit (or data repository) for storing data. The computer system 10101 can be operatively coupled to a computer network (“network”) 10130 with the aid of the communication interface 10120. The network 10130 can be the Internet, an internet and/or extranet, or an intranet and/or extranet that is in communication with the Internet. The network 10130 in some cases is a telecommunication and/or data network. The network 10130 can include one or more computer servers, which can enable distributed computing, such as cloud computing. The network 10130, in some cases with the aid of the computer system 10101, can implement a peer-to-peer network, which can enable devices coupled to the computer system 10101 to behave as a client or a server.

The CPU 10105 can execute a sequence of machine-readable instructions, which can be embodied in a program or software. The instructions can be stored in a memory location, such as the memory 10110. The instructions can be directed to the CPU 10105, which can subsequently program or otherwise configure the CPU 10105 to implement methods of the present disclosure. Examples of operations performed by the CPU 10105 can include fetch, decode, execute, and writeback. The CPU 10105 can be part of a circuit, such as an integrated circuit. One or more other components of the system 10101 can be included in the circuit. In some cases, the circuit is an application specific integrated circuit (ASIC).

The storage unit 10115 can store files, such as drivers, libraries and saved programs. The storage unit 10115 can store user data, e.g., user preferences and user programs. The computer system 10101 in some cases can include one or more additional data storage units that are external to the computer system 10101, such as located on a remote server that is in communication with the computer system 10101 through an intranet or the Internet.

The computer system 10101 can communicate with one or more remote computer systems through the network 10130. For instance, the computer system 10101 can communicate with a remote computer system of a user (e.g., a Smart phone installed with application that receives and displays results of sample analysis sent from the computer system 10101). Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants. The user can access the computer system 10101 via the network 10130.

Methods as described herein can be implemented by way of machine (e.g., computer processor) executable code stored on an electronic storage location of the computer system 10101, such as, for example, on the memory 10110 or electronic storage unit 10115. The machine executable or machine readable code can be provided in the form of software. During use, the code can be executed by the processor 10105. In some cases, the code can be retrieved from the storage unit 10115 and stored on the memory 10110 for ready access by the processor 10105. In some situations, the electronic storage unit 10115 can be precluded, and machine-executable instructions are stored on memory 10110.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 10101, can be embodied in programming. Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which can provide non-transitory storage at any time for the software programming. All or portions of the software can at times be communicated through the Internet or various other telecommunication networks. Such communications, for example, can enable loading of the software from one computer or processor into another, for example, from a management server or host computer into the computer platform of an application server. Thus, another type of media that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as media bearing the software. As used herein, unless restricted to non-transitory, tangible “storage” media, terms such as computer or machine “readable medium” refer to any medium that participates in providing instructions to a processor for execution.

Several machine readable media, for example a as computer-executable code, may be used. Such media can be of many forms, including but not limited to, a tangible storage medium, a carrier wave medium or physical transmission medium. Non-volatile storage media include, for example, optical or magnetic disks, such as any of the storage devices in any computer(s) or the like, such as can be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media can be involved in carrying one or more sequences of one or more instructions to a processor for execution.

The computer system 10101 can include or be in communication with an electronic display 10135 that comprises a user interface (UI) 10140 for providing, for example, results of sample analysis, such as, but not limited to graphic showings of relative and/or absolute amounts of cell-free nucleic acids from different tissues, control or reference amount of cell-free nucleic acids from certain tissues, comparison between detected and reference amounts, and readout of presence or absence of cancer metastasis. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 10105. The algorithm can, for example, control sequencing of the nucleic acid molecules from a sample, direct collection of sequencing data, analyzing the sequencing data, or determining a classification of pathology based on the analyses of the sequencing data.

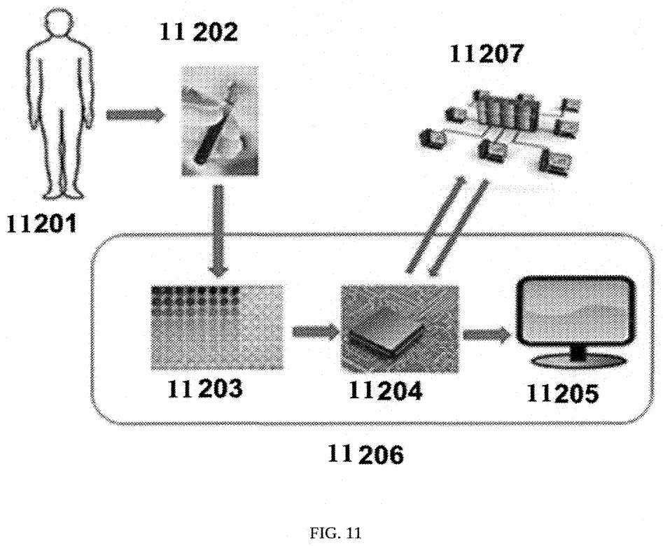

In some cases, as shown in FIG. 11, a sample 11202 can be obtained from a subject 11201, such as a human subject. A sample 11202 can be subjected to one or more methods as described herein, such as performing an assay 11203. In some cases, an assay can comprise hybridization, amplification, sequencing, labeling, epigenetically modifying a base, or any combination thereof. One or more results from a method can be input into a processor 11204. One or more input parameters such as sample identification, subject identification, sample type, a reference, or other information can be input into a processor 11204. One or more metrics from an assay can be input into a processor 11204 such that the processor can produce a result, such as a classification of pathology (e.g., diagnosis) or a recommendation for a treatment. A processor can send a result, an input parameter, a metric, a reference, or any combination thereof to a display 11205, such as a visual display or graphical user interface. A processor 11204 can (i) send a result, an input parameter, a metric, or any combination thereof to a server 11207, (ii) receive a result, an input parameter, a metric, or any combination thereof from a server 11207, (iii) or a combination thereof. In some embodiments, an integrated diagnosis system 11206 may comprise the assay 11203, processor 11204, and display 11205 in a compact device and perform the diagnosis method automatically.

Aspects of the present disclosure can be implemented in the form of control logic using hardware (e.g., an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments described herein using hardware and a combination of hardware and software.

Any of the software components or functions described in this application can be implemented as software code to be executed by a processor using any suitable computer language such as, for example, Java, C, C++, C#, Objective-C, Swift, or scripting language such as Perl or Python using, for example, conventional or object-oriented techniques. The software code can be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium can be any combination of such storage or transmission devices.

Such programs can also be encoded and transmitted using carrier signals adapted for transmission via wired, optical, and/or wireless networks conforming to a variety of protocols, including the Internet. As such, a computer readable medium can be created using a data signal encoded with such programs. Computer readable media encoded with the program code can be packaged with a compatible device or provided separately from other devices (e.g., via Internet download). Any such computer readable medium can reside on or within a single computer product (e.g., a hard drive, a CD, or an entire computer system), and can be present on or within different computer products within a system or network. A computer system can include a monitor, printer, or other suitable display for providing any of the results mentioned herein to a user.

Any of the methods described herein can be totally or partially performed with a computer system including one or more processors, which can be configured to perform the steps. Thus, embodiments can be directed to computer systems configured to perform the steps of any of the methods described herein, with different components performing a respective steps or a respective group of steps. Although presented as numbered steps, steps of methods herein can be performed at a same time or in a different order. Additionally, portions of these steps can be used with portions of other steps from other methods. Also, all or portions of a step can be optional. Additionally, any of the steps of any of the methods can be performed with modules, units, circuits, or other approaches for performing these steps.

### Examples of Analytics Systems

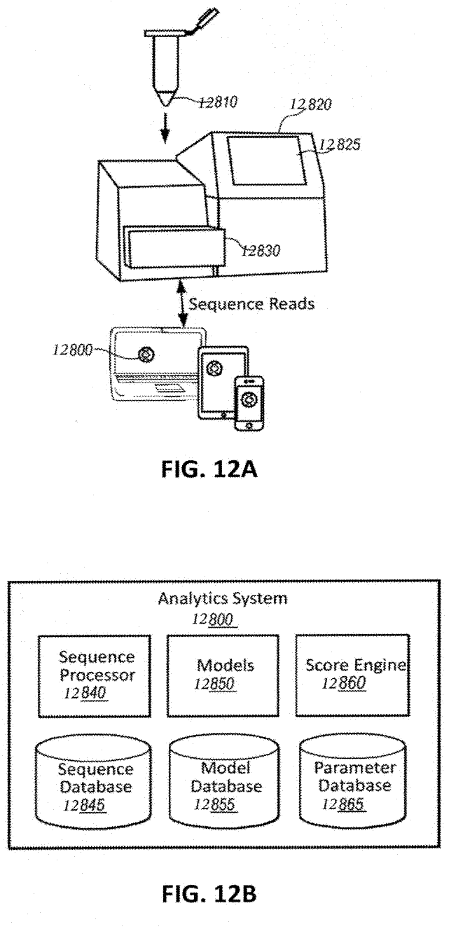

FIG. 12A is a flowchart of systems and devices for sequencing nucleic acid samples according to one embodiment. This illustrative flowchart includes devices such as a sequencer 12820 and an analytics system 12800. The sequencer 12820 and the analytics system 12800 may work in tandem to perform one or more steps in the processes described herein.

In various embodiments, the sequencer 12820 receives an enriched nucleic acid sample 12810. As shown in FIG. 12A, the sequencer 12820 can include a graphical user interface 12825 that enables user interactions with particular tasks (e.g., initiate sequencing or terminate sequencing) as well as one more loading stations 12830 for loading a sequencing cartridge including the enriched fragment samples and/or for loading necessary buffers for performing the sequencing assays. Therefore, once a user of the sequencer 12820 has provided the necessary reagents and sequencing cartridge to the loading station 12830 of the sequencer 12820, the user can initiate sequencing by interacting with the graphical user interface 12825 of the sequencer 12820. Once initiated, the sequencer 12820 performs the sequencing and outputs the sequence reads of the enriched fragments from the nucleic acid sample 12810.

In some embodiments, the sequencer 12820 is communicatively coupled with the analytics system 12800. The analytics system 12800 includes some number of computing devices used for processing the sequence reads for various applications such as assessing methylation status at one or more CpG sites, variant calling or quality control. The sequencer 12820 may provide the sequence reads in a BAM file format to the analytics system 12800. The analytics system 12800 can be communicatively coupled to the sequencer 12820 through a wireless, wired, or a combination of wireless and wired communication technologies. Generally, the analytics system 12800 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to determine alignment position information. Alignment position may generally describe a beginning position and an end position of a region in the reference genome that corresponds to a beginning nucleotide based and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 12800 may label a sequence read with one or more genes that align to the sequence read. In one embodiment, fragment length (or size) is determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome represent the likely location within the reference genome to which the nucleic acid fragment corresponds. In one embodiment, the read pair R_1 and R_2 can be assembled into a fragment, and the fragment used for subsequent analysis and/or classification. An output file having SAM (sequence alignment map) format or BAM (binary) format may be generated and output for further analysis.

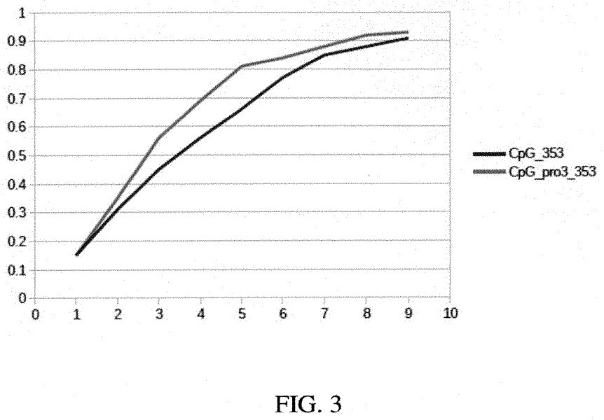

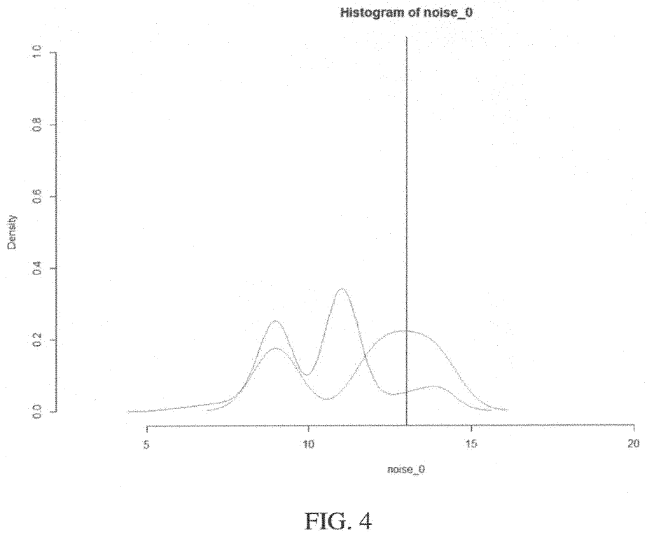

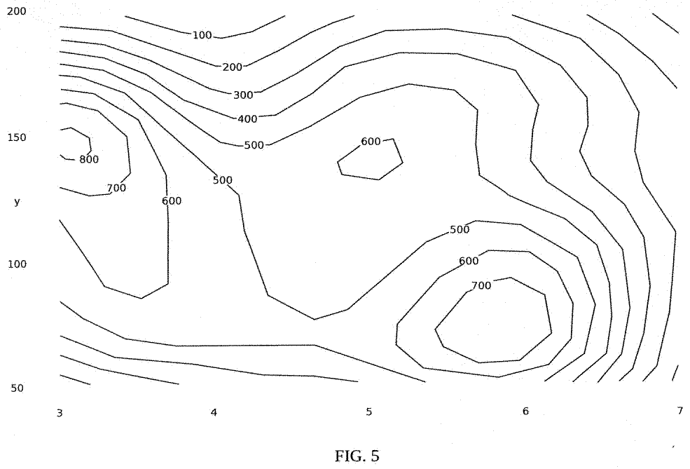

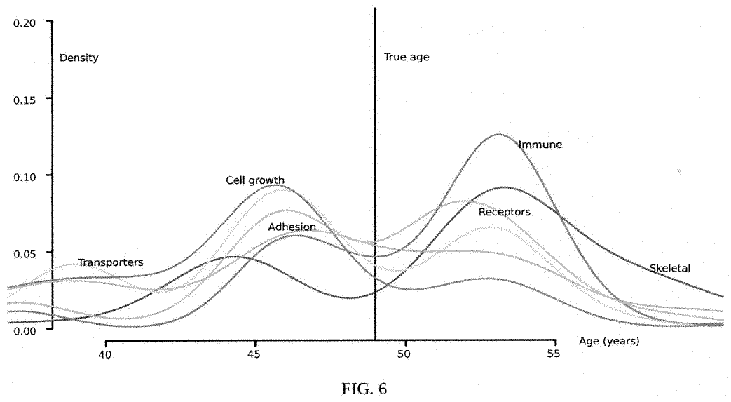

Referring now to FIG. 12B, FIG. 12B is a block diagram of an analytics system 12800 for processing DNA samples according to one embodiment. The analytics system implements one or more computing devices for use in analyzing DNA samples. The analytics system 12800 includes a sequence processor 12840, sequence database 845, model database 12855, models 12850, parameter database 12865, and score engine 12860. In some embodiments, the analytics system 12800 performs one or more steps in the processes 300 of FIG. 3A, 340 of FIG. 3B, 400 of FIG. 4, 500 of FIG. 5, 600 of FIG. 6A, or 680 of FIG. 6B and other process described herein.

The sequence processor 12840 generates methylation state vectors for fragments from a sample. At each CpG site on a fragment, the sequence processor 12840 generates a methylation state vector for each fragment specifying a location of the fragment in the reference genome, a number of CpG sites in the fragment, and the methylation state of each CpG site in the fragment whether methylated, unmethylated, or indeterminate via the process 300 of FIG. 3A. The sequence processor 12840 may store methylation state vectors for fragments in the sequence database 12845. Data in the sequence database 12845 may be organized such that the methylation state vectors from a sample are associated to one another.

Further, multiple different models 12850 may be stored in the model database 12855 or retrieved for use with test samples. In one example, a model is a trained cancer classifier for determining a cancer prediction for a test sample using a feature vector derived from anomalous fragments. The training and use of the cancer classifier is discussed elsewhere herein. The analytics system 12800 may train the one or more models 12850 and store various trained parameters in the parameter database 12865. The analytics system 12800 stores the models 12850 along with functions in the model database 12855.

During inference, the score engine 12860 uses the one or more models 12850 to return outputs. The score engine 12860 accesses the models 12850 in the model database 12855 along with trained parameters from the parameter database 12865. According to each model, the score engine receives an appropriate input for the model and calculates an output based on the received input, the parameters, and a function of each model relating the input and the output. In some use cases, the score engine 12860 further calculates metrics correlating to a confidence in the calculated outputs from the model. In other use cases, the score engine 12860 calculates other intermediary values for use in the model.

### ADDITIONAL EXAMPLES

The following examples are intended to illustrate details of the invention, without thereby limiting it in any manner. As described in the examples below, the use of PLGA can significantly provide high decitabine loading, a limited burst, sustained delivery in the vitrous and retina, does not result in inflammation of the eye following administration, and additional features as disclosed herein.

### Example 1

In this prophetic example, an epigenetic interface analyzes a subject's genome from a number of non-intrusive sources including saliva or blood samples. Neural networks will then process the subject's genome and discover characteristics which correlate with particular phenotypes and physiological parameters. These findings are then compared to the general population to reveal the subject's genome's personal performance. A physician or health-care individual provides the subject with a personalized health assessment. The physician or health-care individual further prescribes or advises the a medical treatment course to address the individual's personalized health assessment.

### Example 2

Using a “replacement by synonymity” approach, surprisingly it was identified that a set of 353 CpG islands where >50% of the CpG islands had been replaced by novel, previously unreported CpG islands. Introduction of the novel CpG islands in the in-house data set significantly lowered the prevalence of known CpG islands without compromising prediction performance. Even more surprisingly, some neural network designs even favored the substituted data set, indicating a specific role for these CpG islands in optimizing age prediction.

### Example 3—KS 15

Transmembrane signaling receptor activity was investigated. The check enrichment is discussed in Table 1.

TABLE 1

| GO biological process | | | | Fold | | Raw P | |
|---|---|---|---|---|---|---|---|
| complete | # | # | expected | enrichment | +/− | value | Δ FD |
| Signal transduction | 5156 | 164 | 70.47 | 2.33 | + | 2.72E−31 | 4.33E−27 |
| Signaling | 5518 | 166 | 75.42 | 2.20 | + | 6.77E−29 | 5.38E−25 |
| Cell Communication | 5609 | 166 | 76.67 | 2.17 | + | 3.76E−28 | 1.99E−24 |
| Cellular response to stimulus | 6808 | 178 | 93.05 | 1.91 | + | 2.61E−24 | 1.04E−20 |
| Response to stimulus | 8545 | 197 | 116.8 | 1.69 | + | 1.92E−21 | 6.12E−18 |
| G-protein-coupled receptor | 1324 | 65 | 18.10 | 3.59 | + | 4.13E−19 | 1.10E−15 |
| signaling pathway | | | | | | | |
| Cell surface receptor | 2523 | 85 | 34.49 | 2.46 | + | 4.4E−15 | 8.04E−12 |
| signaling pathway | | | | | | | |
| Regulation of biological | 11873 | 226 | 162.29 | 1.39 | + | 4.4E−15 | 9.17E−12 |
| process | | | | | | | |
| Regulation of cellular | 11311 | 216 | 154.60 | 1.40 | + | 1.15E−13 | 2.03E−10 |
| process | | | | | | | |
| Response to chemical | 4415 | 113 | 60.35 | 1.87 | + | 2.10E−12 | 3.34E−09 |
| Biological regulation | 12544 | 227 | 171.46 | 1.32 | + | 4.31E−12 | 6.24E−09 |
| Cellular process | 15626 | 256 | 213.58 | 1.20 | + | 4.74E−10 | 6.28E−07 |
| Regulation of locomotion | 922 | 41 | 13.56 | 3.02 | + | 5.53E−.10 | 6.77E−07 |
| Regulation of cellular | 1039 | 42 | 14.20 | 2.9 | + | 6.32E−10 | 7.17E−07 |
| component movement | | | | | | | |

### Example 4—KS 16

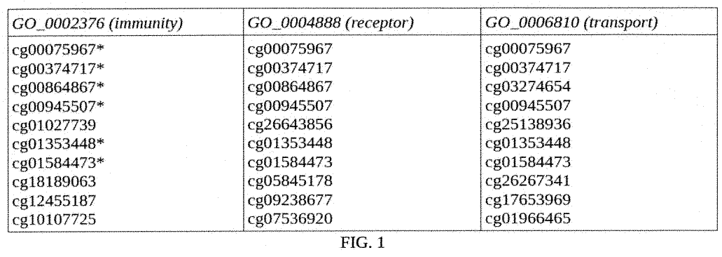

Steve Horvath presented a list of 353 CpG islands in his paper, Genome Biol, 2013; 14(10):R115. He describe predictive performance as a set error of 3.6 years, indicating that DNAm age differs by less than 3.6 years in 50% of subjects. Graphs of error cutoff vs percentage of test cases that meet that cutoff were plotted (FIG. 1.). He referred to this measure as (median) ‘error’—that is the median absolute difference between DNAm age and chronological age. It has been identified that relying on the total sum or error in a test set of 100 individuals, frequently trying to keep this measure under 300 would be desirable. Depending on the tissue, he reported Pearson correlation coefficients ranging from 0.89 to 0.97. His CpG island list can be found in Supplementary File 3 of his paper, and starts with: cg00075967, cg00374717, cg00864867. The following three data sets were used in this example: GSE27097 (leukocytes from autism sibs, children from Atlanta, Ga.), GSE41037 (whole blood, adults from Los Angeles, Calif.), and GSE42861 (whole blood, adults from Shanghai, China) because they are large, and complement each other in their coverage of different age groups. The first step in the data processing is using a Perl script that extracts lines from the series matrix files that contain the relevant CpG islands only. Using the int-run programmatic interface 0.1, a standard benchmark was performed of the 353 published CpG islands vs. something called the “pro3 set”, an in-house version of this set, where 50% of the islands have been replaced by de-novo islands. A Python script trains a Keras model using 4×95-sized Dense layers for 73 epochs.

FIG. 13 illustrates an exemplary method of feature selection from data, implemented by the feature selection module 100 described in connection with FIG. 9. As described above, the raw data set 10, comprising raw data of a plurality of samples, is inputted into the feature selection module 100. The feature selection module 100 then performs step 101. In step 101, for each “Horvath learned feature” (i.e., one of the 353 CpG islands discovered by Horvath which, in combination, having a good predicting power of an individual's age), the computer system automatically searches and attempts to find a “synonymous feature” from the rest of the CpG islands. The “synonymous feature” may be required to be highly correlated with the Horvath learned feature in the plurality of samples and is located on a different gene. If a synonymous feature is found, the computer system stores the synonymous feature.

After finding as many synonymous features as possible, the feature selection module 100 then performs step 102. In step 102, for each sample, the computer system generates a modified feature list by replacing the Horvath learned feature with the found synonymous feature. The feature selection module 100 then outputs a training data set 20, which comprises the modified feature lists of the plurality of samples.

Using a higher order GO term (e.g., GO:0008152, metabolic process) as a filter, and BioMart to select Gene names from Ensembl Genes 100, Human genes (GRCh38.p13), retrieving only the gene name as feature, and filtering for redundant results, resulted in a per-filtering gene count of 11.742. Next, a Perl script to grep cg IDs was used to identify gene names from GPL13534-11288.txt, a mapping between CpG islands and locations etc. The cg IDs were used to grep full lines from GSE27097_series_matrix.txt. A second Perl script was used to replace each cg ID in the Horvath 353 CpG list by a cg ID that maps to the current GO term, calculating Pearson correlation and heuristically selecting the best synonymous CpG island as a substitution. A third Perl script was used to filter the replacement CpG list, retaining the original CpG island if the replacement island had a Pearson 1 correlation lower than e.g. 0.75 compared to the original CpG island, and also excluding any replacement CpG islands that do not map to all of GSE27097, 41037, or 42861, while maintaining a replacement frequency of greater than 50%, preferably higher. A fourth Perl script was used to decode the final cg ID list as common gene names, which can be confirmed for enrichment as measured by FDR on the Gene Ontology. Following this workflow, stable CpG island lists were created that perform better than 300 errors per 100 test cases, and displaying FDRs for enriched GO terms in the e-20, e-30, or e-40 range.

Using stochastic noise, a Perl script was used to introduce noise at a certain user defined level (0.01-0.2), or in increments of $noise level=($m*0.025), where $m ranges from 0-10. The noise is introduced to the CpG methylation probability array for a selected individual using the formula ($line[$ind]+rand($noise_level)−($noise_level/2)). Each noised replicate is used as input to the machine learning process, and the results are plotted as a probability distribution in R studio.

Using a parameter scanning approach in Python, three random numbers were generated num1=random.randint(3, 7), num2=random.randint(50, 200), num3=random.randint(50, 100). These numbers correspond to the number of dense layers to be added to the Keras model, the size of each layer, and the number of epochs of training to be performed. A sample run of 5 epochs is performed to determine if the current setting favors CPU or GPU usage. Commonly, the product of num1 and num2 is also recorded as a measure of the number of trainable parameters.

### Example 5—KS 18

Horvath presented a list of 353 CpG islands in his paper, Genome Biol, 2013; 14(10):R115, describing predictive performance as a set error of 3.6 years, indicating that DNAm age differs by less than 3.6 years in 50% of subjects (depending on the tissue, data selection etc., performance could be worse than the aforementioned level). A graph of error cutoff vs percentage of test cases that meet that cutoff was plotted (FIG. 1.). Other authors have referred to this measure as (median) ‘error’—that is the median absolute difference between DNAm age and chronological age. The total sum or error in a test set of 100 individuals can be used, ideally trying to keep this measure under 300-400 units.

A look up table may be used instead of machine learning or regression., but with very large look up tables, results are not optimal. The performance of the machine learning is similar to that of published regression models, but offers other advantages including ease of CpG list manipulation. Performance is at the limit that can be achieved and that the variability depends on the biology and on the variable aging of different genes and systems. Earlier abandoned approaches have included bisection and bins approaches using a consensus of many predictions, but it can be better to not bin the data, and to not use a consensus approach.

Rather, each array of probabilities of methylation with the age of the subject is compiled and this unprocessed data is fed into the deep learning workflow. The deep learning workflow presented here was initially based on softmax regression used to classify handwritten digits. Depending on the tissue, other authors (Genome Biol, 2013; 14(10):R115) is reporting Pearson correlation coefficients ranging from 0.89 to 0.97, using a particular CpG island list that can be found in Supplementary File 3 (of Genome Biol, 2013; 14(10):R115), and starts with: cg00075967, cg00374717, cg00864867. In this prophetic example, the following three data sets are used: GSE27097 (leukocytes from autism sibs, children from Atlanta, Ga.), GSE41037 (whole blood, adults from Los Angeles, Calif.), and GSE42861 (whole blood, adults from Shanghai, China), because they are large, and collectively complement each other in their coverage of different age groups. The first step in the data processing is using a Perl script that extracts lines from the “series matrix” files that contain the relevant CpG islands only. Using the “int-run programmatic interface 0.1”, a standard benchmark of the 353 published CpG islands vs. something called the “pro3 set” was performed, an in-house version of this set, where 50% of the islands have been replaced by de-novo islands.

In short, the int-run programmatic interface 0.1 does following: A Python script trains a Keras model using 4×95-sized Dense layers for 73 epochs. Further details are outlined below:

Input may be reformatted as a square shape with some empty cells (20×20 dim). A random sample of 100 from the concatenated file were used. This could be different each time, and could certainly arbitrarily affect performance of an evaluation run. Temporary test set selections used a time stamp in seconds from a point back in time (used as temporary file name) and labeled. The test set selection can be in srandmode, where the selection is the same each time, in case of wanting to compare the same selection between e.g. different CpG island lists or different optimized parameters. The Keras model contains 3 invariable initial layers, a central portion with a variable number of N×M-sized dense layers, and 2 invariable final layers. The first invariable initial layer consists of a Conv2D layer, sized 100, with a 3×3 kernel size and a 20×20 input shape. The second invariable initial layer consists of a MaxPooling 2D layer with a 2×2 pool size. The third invariable initial layer (or layer operation) is a flattening operation, flattening the 2-d layer for fully connected layers. These initial layers are followed by a N×M-sized Dense layers, using tf.nn.rely as the activation function. Finally, before the compilation of the model, there is a Dropout layer (parameter 0.2). This is followed by the final Dense, 100—sized layer, using the tf.nn.softmax (Softmax) activation function. Compilation is achieved using a sparse categorical cross entropy, using accuracy metrics. A choice between whether to use CPU or GPU for the current parameters (N layers, M-sized layers, E # of epochs) is estimated from timing a 5-epochs long non-verbose test run using CPU and GPU. In some embodiments of the Python interface, the trained model can be saved and re-loaded, depending on the application.

A higher order GO term (e.g., GO:0008152, metabolic process) as a filter, and BioMart to select Gene names from Ensembl Genes 100, Human genes (GRCh38.p13), retrieving only the gene name as feature, and filtering for redundant results, resulted in a per-filtering gene count of 11.742. GO term enriched CpG island lists tend to perform slightly worse than CpG island lists that are merely substituted for performance tying to substitute more than 50% of published CpG islands were identified. However, for some GO categories, including metabolism, that are very large, sometimes performance is worse than for smaller more specialized GO categories.

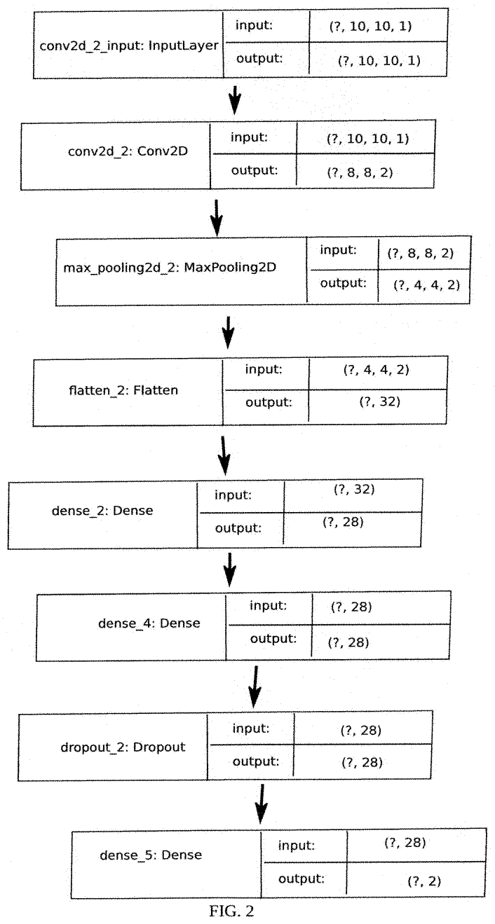

Next, a Perl script to grep cg IDs was used to identify gene names from GPL13534-11288.txt, a mapping between CpG islands and locations etc. The cg IDs are in turn used to grep full lines from GSE27097_series_matrix.txt. This is essentially creating a subset of the input file (series matrix file). A second Perl script was used to replace each cg ID in the public 353 CpG list by a cg ID that maps to the current GO term, calculating Pearson correlation and heuristically selecting the best synonymous CpG island as a substitution. A third Perl script was used to filter the replacement CpG list, retaining the original CpG island if the replacement island had a Pearson correlation lower than e.g. 0.75 compared to the original CpG island, and also excluding any replacement CpG islands that do not map to all of GSE27097, 41037, or 42861 data sets, while maintaining a replacement frequency of greater than 50%, preferably higher. A fourth Perl script is used to back-decode the final cg ID list as common gene names, which can be confirmed for enrichment as measured by FDR on the Gene Ontology. Following this workflow, stable CpG island lists were created that perform better than 300 errors per 100 test cases, and displaying FDRs for enriched GO terms in the e-20, e-30, or e-40 range (FIG. 2). The final step in this workflow would be to test initial performance of the newly created CpG island list, output a probability distribution for test individuals (using the srand version of the programmatic interface to enforce a particular arbitrary test group selection), as well as doing a parameter scan with the new CpG island list to find optimal parameters for future use (FIG. 4).

FIG. 14 illustrates an exemplary method of generating diagnosis output, implemented by the diagnosis module 400 described in connection with FIG. 9. In step 401, the diagnosis module 400 takes the new data 40 of a patient and the final model (F) 35 as inputs. The new data 40 of the patient comprises the same kind of bioinformatic data and biological properties, as used in the training data set 20 and testing date set 30, of a sample from the patient. In one embodiment, the new data 40 of the patient comprises measured patient data (X) and patient chronological age (A). In some embodiments, the measured patient data (X) is the methylation probability of CpG islands as a function of chromosomal location of the CpG islands of the sample from the patient.

In step 402, the diagnosis module 400 then creates a synthetic data (Y) by adding a noise (δ) to the measured patient data (X), i.e., Y=X+δ. Both X and δ may be vector or tensor depending on the nature of the data. In particular, the noise (δ) is generated by a predetermined random number generator. In step 403, the diagnosis module 400 then calculates a predicted age (B) from the synthetic data (Y), by evaluating the synthetic data (Y) on the final model (F), i.e., B=F(Y). The steps 402 and 403 are repeated a plurality of times in order to generate a plurality of predicted ages (B's).

In step 404, the diagnosis module 400 then summarizes the plurality of predicted ages (B's) as a probability density function p(B). In step 405, the diagnosis module 400 then identifies a peak (having age value C) of the probability density function p(B), and retrieves a corresponding synthetic data (Z) such that C=F(Z).

In step 406, the diagnosis module 400 then finds a noise (ζ) such that F(Z+ζ)=A, the patient chronological age. In step 407, the diagnosis module 400 then subtracts the noise (ζ) from X, and reports (X−ζ) as part of the diagnosis output 45. The diagnosis module 400 may also attribute the peak (C) to certain features e.g., certain CpG islands, and reports such features as part of the diagnosis output 45. Step 405 through step 407 may be repeated for all the local peaks shown in p(B).

Using a noise approach, probability distributions of age predictions for different biological systems, as opposed to presenting age predictions as a mere number were compared. The idea of introducing noise to machine learning input data is not a new one, but new application areas including applying noise to an incorrect prediction multiple times until a correct prediction is arrived. This can be repeated multiple times and may show that different offsets can correct the answer, but can also be used to attribute “error” to a subset of the CpG island list and the associated genes. A further application area was explored was the use of noise of different equally spaced magnitudes to generate two-dimensional peak landscapes. A decision tree was proposed to correct poor predictions using such complex landscapes where the true age frequently manifests itself at a certain noise level. In the end, the most promising application area of noise in this field, was believed to be novel, was the general concept of presenting age predictions as probability distributions. Essentially using an R script to convert a histogram to a probability distribution was identified. By calculating multiple such distributions and juxtaposing the results between different CpG island lists representing different biological systems, was compared against senescence processes across biological systems.

In short, using stochastic noise, a Perl script was used to introduce noise at a certain user defined level (0.01-0.2), or in increments of $noise_level=($m*0.025), where $m ranges from 0-10. The noise is introduced to the CpG methylation probability array for a selected individual using the formula ($line[$ind]+rand($noise_level)−($noise_level/2)). Each noised replicate is used as input to the machine learning process, and the results are plotted as a probability distribution in R studio (FIG. 3).

Using parameter scanning, the performance of Go-term enriched CpG island lists can be optimized for prediction. Early on, an intriguing inverse relationship between the machine learning parameters (M*N (variable layer size of trainable parameters) and number of epochs of training was identified. It is important to match composite M*N data points with their M and N values when compare M and N, as well as M*N vs epochs (E). Different parameters were selected when compared bottom and top results from a parameter scan run, but it was decided to link specific parameters more to the current biological bias of each CpG island list was also considered. An important development has been the concept of plotting 2-d density of parameter scan parameters and fitting a topographical contour to such landscapes, and comparing such contours between biological systems studied. Using a parameter scanning approach in Python, three random numbers are generated num1=random.randint(3, 7), num2=random.randint(50, 200), num3=random.randint(50, 100). These numbers correspond to the number of dense layers to be added to the Keras model, the size of each layer, and the number of epochs of training to be performed. A sample run of 5 epochs is performed to determine if the current setting favors CPU or GPU usage. Commonly, the product of num1 and num2 is also recorded as a measure of the number of trainable parameters (FIG. 4).

### Example 6—Utility of Variational Auto Encoders in a Points-Based Methyl-HealthIndex

The idea of a points-based HealthIndex, grouped by clinical categories (and here exemplified by Lupus data), relates to age prediction, where the difference between chronological age and “biological” age is used as a healthspan indicator. As opposed to age, which is one of the most recurring methylation data annotations, many public data sets are linked to a clinically relevant, usually binary, annotation field that is frequently easy to predict within the “native” data. The concept of the HealthIndex is then to combine and add up positive and negative health points from many predictors, each trained on separate data sets. A technical problem is that different data sets may use different origin tissues, different data collection platforms, and so forth, and there may not be an easy way to validate any predictions made outside the native data. It was demonstrated here several points about non-native predictions made towards the goal of developing the health index, using Lupus data for illustration purpose. The issues discussed and dissected are the following: A) how to make a binary prediction within native data, B) if it cannot be verified from predictions in non-native data, what other approaches can be taken to evaluate such predictions. In particular, those approaches include: i) testing whether non-native predictions are robust and diverse, ii) using VAEs (variational auto encoders, not the decoder part) to infer, visualize and confirm supervised predictions (and detect anomalies, sometimes using ggplot or pyplot), and iii) directly filtering non-native predictions using VAEs that have been parameter scanned and optimized on native data.

Lupus is an auto-immune disorder that is more common in certain demographic groups (PMID: 29361205). The symptoms of lupus include skin problems such as a butterfly-shaped rash on the face, and even kidney failure. The disease is believed to have a strong methylation component, making it very easy to predict (PMID: 23950730).

The comparison of chronological age, and age predicted using methylation data (mAge), is building on the idea that DNA methylation represses transcription, and that certain CpG islands (the dinucleotide pair in DNA that can be methylated) display a probability of methylation that strongly correlates with gender, development, age, and also health.

The fact that many public methylation data sets are annotated with age, gender, and sometimes ethnicity, make age predictions particularly easy to verify, even though different data sets may use different tissues, such as whole blood or saliva. Many labs have developed certain age prediction methods, including the “GrimAge” remaining lifespan index of the Horvath lab at UCLA (PMID: 30669119). These methods have also been commercialized (PMID: 32791973), sometimes sold as an independent service or sold in conjunction with anti-aging supplements, such as nicotinamide mononucleotide (NMN), and similar compounds.

The current state of the art prediction accuracy can be said to be within ˜3 years, where the discrepancy is largely attributable to each individual's biologically unique senescence process, and to a lesser extent, prediction error. While some methods have made predictions using only a few CpG islands, recent methods have first shifted towards using regression models, and more recently towards deep learning methods, such as using Tensorflow and the Keras package in a Python environment, including the use of Variational Auto Encoders (VAEs), as in the MethylNet method (PMID: 32183722). VAEs, which have sometimes been used for noise reduction, take the raw data and transform it using a few dense Keras layers to an internal, sometimes visualizable dimension. From there, each point in the inner dimension can be used as a starting point to generate “fabricated” methylation data.

Here, a deep learning predictor was developed for lupus and test it on native data. It has been shown that the predictor can be successfully applied on non-native data, using the criteria of diversity and robustness rather than “accuracy”. This example is provided to illustrate how a points-based HealthIndex could be incrementally built up, in the wider general context of age and healthspan predictions.

In particular, it has been shown how unsupervised VAEs can be used to visualize, and confirm the supervised predictions in a 2-d transform space. It has also been shown how parameter scanning can be used to find appropriate VAE parameters, including number and size of dense layers, dimensionality of transform space, epochs of training, and batch size, to optimize verifiable predictions on native data. Furthermore, the optimized VAE settings was applied on non-native data to filter the robust and diverse predictions.

In one embodiment, disclosed methods may include:

Step 1. Prediction on native data: Start with a public data set, GSE59250, and extract the “label” information (whether a subject has lupus, or not). There were 434 subjects (the origin tissue is either B-cells, T-cells or monocytes), and 482.421 CpG islands. A Perl script was used to look for CpG islands that correlate (to some extent) with the labeling. 353 (non-redundant) CpG islands were extracted that show a correlation with the label greater than 0.1. The data was split into 100 randomly selected test subjects, and 334 training subjects (the remainder). The data was reformatted into Python input files, where the methylation probability values of the 353 CpG island were fitted into 20×20-sized padded matrices.

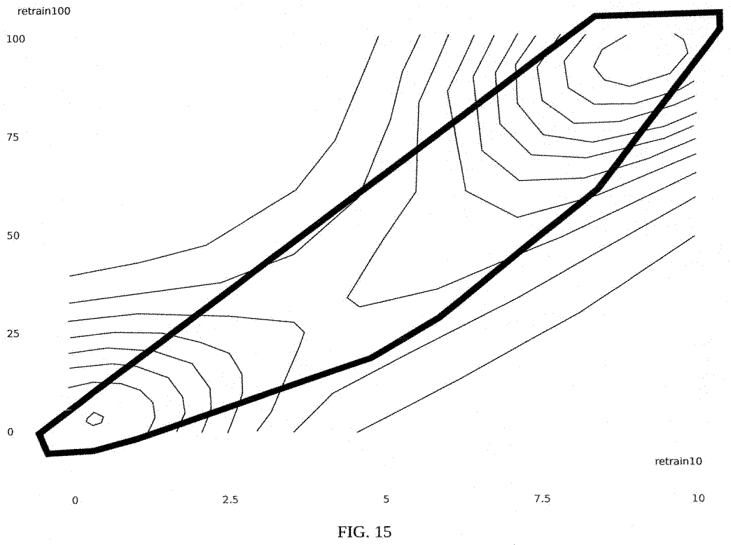

Step 2. Prediction on non-native data: While the prediction performance on native data was good (see Results #1 below), the challenge comes when applying the model on non-native data, which could include data from a different tissue or data generation platform. To develop a points-based HealthIndex, where the true answer may not be known, there were two things to look for: “diverse and repeatable” predictions. For testing on non-native data, a different data set, GSE40279, was frequently used which comes from whole blood and contains 656 middle aged subjects, and contains 470.043 CpG islands (PMID: 23177740). By summing the positive supervised predictions from 10 or 100 retrainings, an average prediction outcome was determined and visualized using e.g. ggplot (see FIG. 15). As shown in FIG. 15, retraining the model and counting the number of positive predictions from 10 vs. 100 consecutive retrainings reveal that the predictions are “robust”, even though a “correct” answer is not necessarily known.

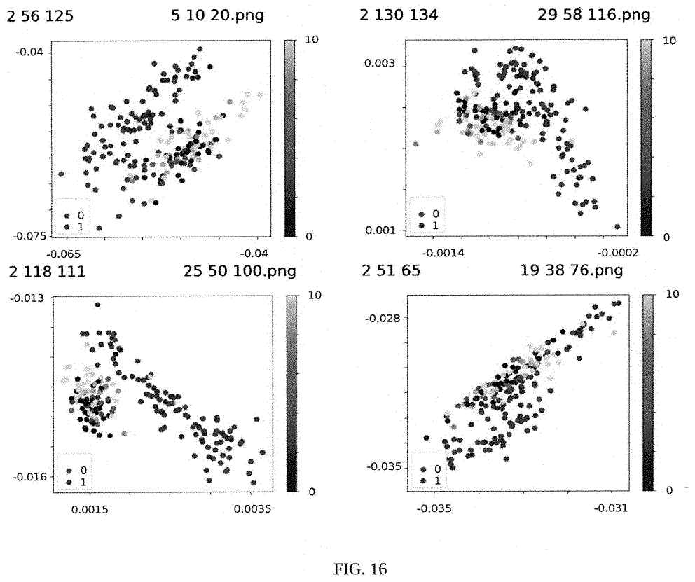

Step 3. Visualizing supervised prediction using VAE encoder transform space: Using a variational autoencoder (VAE), it can display the location of non-native data points in a 2-d transform space by training the VAE to recreate or filter native data, and then applying merely the encoder on the non-native data to map the coordinates of those points alongside the native data in the low dimensional space, using e.g. PyPlot to color code the findings (see FIG. 16). As shown in FIG. 16, different VAE settings (transform dimension size, epochs of training, batch size, and triple dense layer dimensions) can suggest a non-supervised categorization of the non-native data points (color coded from 0-10 (blue→green→yellow) based on their supervised learning propensity to be, in this case, “lupus like” data); the actual lupus cases are the “green” dots (marked by 1 in the corner legend).

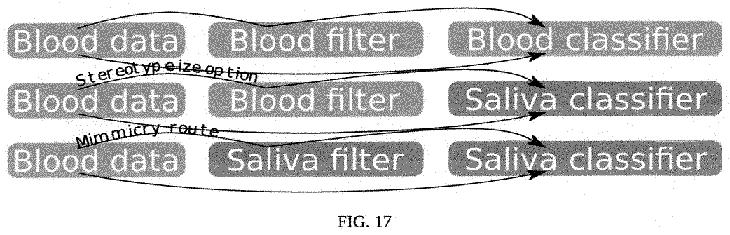

Step 4. Using VAEs to filter input data: a classifier, a data set and a filter were used. The idea was that the filter would improve performance on a dataset when a classifier was used that has been trained on a different data set. The approach would be to set up some criteria, such as maximizing correlation between actual and predicted age (with or without filtering), or studying total sum error of 100 test cases and scan for sets of filter parameters that give a strong filter. The parameter set representing each filter are given as 3×3 numbers, where the first three numbers denote the data sets used for filter, classifier, and input data. The second triplet of parameters denote the Z-dim (dimensionality of transform space, only visualizable if set to 2), the batch size, and the number of epochs of training. The final set of numbers are the sizes used for three dense Keras layers used in the filter. Such approaches may be difficult to systematically evaluate because each retraining will give a different result. To begin with, using a classifier from a different data set, performance was much lower. While it has been seen that some success with some types of VAE filters, the final R (correlation coefficient) between true and predicted age may not be high in cases where the filter appears to provide some benefit. A distinction can be made between “mimicking” and “stereotype” filter, based on whether the VAE filter is trained on the data the classifier was trained on, or trained on the input data itself, respectively (see FIG. 17). Another approach would be to train two different VAEs (encoder and decoder) on the native and non-native data, and remap the corresponding areas in the 2-d transform space, i.e. take both VAEs apart and combining an encoder for e.g. blood with a decoder for e.g. saliva in the transform space. An example of a decent filter is a certain “mimicking” and “stereotype” filter that is supposed to translate whole or peripheral blood input to be used in a classifier trained on lung cancer tumor methylation training data (see Results below). FIG. 17 shows parameter scanning (Z-dim, epoch #, batch size, dense layer sizes), for VAE to prepare e.g. blood input data for successful use with a saliva-based classifier. There are two approaches, 1) use of “blood filter” to pre-process blood data (make it more “stereotypical”), or 2) use of “saliva filter” (make it more “saliva like”).

Results

Results #1. Prediction on native data: Using a deep learning model with a 100-sized input layer, 4×95 dense Keras layers, a binary output layer and 73 epochs of training in Tensorflow, excellent classification performance is achieved on native data. Of 100 test cases, only 9 were mispredicted (5 false positives and 4 false negatives), displaying a correlation coefficient of 0.82.

Results #2. Prediction on non-native data: Because the average prediction from 10 retrainings matches the results from 100 retrainings (see FIG. 15), it can be concluded that these predictions are robust, and also diverse, inasmuch as they range from largely positive to largely negative predictions. It can also be concluded that the 10 retrainings may be sufficient to approximate the recurring prediction outcome.

Results #3. Visualizing supervised prediction using VAE encoder transform space: Overall there is a good agreement between the location of the high probability lupus like points (coded yellow) from the supervised prediction (rated from 1-10 positive predictions, and shown as a color gradient in FIG. 16), and the original native data annotation (lupus or not, color coded and labeled as 1 (coded green) or 0), as shown for a number of randomly chosen parameter selections (FIG. 16).

The evident success of this visualization method, which uses the encoder aspect of the trained VAE only, can clearly be used for visualization and anomaly detection in this exercise and would have broad applicability in the development of the novel HealthIndex.

The choice of parameters may be debatable and high dimensional transform space may be better for recreation of data but may not be visualizable. In the first quadrant of FIG. 16, the parameters used were 2, 56, 125 (A1-3) and 5, 10, 20 (B1-3), where A1-3 represent Z-dim, number of epochs, and batch size, while B1-3 represent dimensions of the dense layers in the encoder and decoder respectively.

Results #4. Using VAEs to filter input data: As it has been stated above, the work to deliver strong VAE based filters to make predictions on non-native data. To give a view of what might become possible, and to delineate the disclosed methods and approach, a VAE-based filter defined by 3×3 settings or parameters has been reported herein. This example filter is meant to pre-process blood input data and prepare it in a way such that it can be classified using a set of Keras dense layers trained on lung cancer (LC) data. Regarding the settings and parameters used, LC data (GSE39279) for the classifier of both the “mimicking” and “stereotype” filter has been used, and LC data for the actual filter of the “mimicking” filter has also been used. For the “stereotype” filter, peripheral blood (GSE50660) as both input data and data to train the filter was used. The input data for the “mimicking” filter is whole blood (GSE40279). The second triplet of parameters are (9, 165, 106) and (10, 154, 63) for the “mimicking” and “stereotype” filter, respectively, and the dense layer size parameters are (19, 38, 76) and (46, 92, 194) for the “mimicking” and “stereotype” filter, respectively. Regarding the performance of this “blood→lung cancer data” filter, it can produce runs that display improved correlation between true and predicted age, as well as a reduction in cumulative total error. To give a flavor of what can be achieved under typical circumstances, it may not be uncommon to see situations where the correlation coefficient is unaffected by the application of the filter but the total error is almost halved (the “mimicking” filter), or up to a doubling of the correlation coefficient coupled with some reduction in total error (the “stereotype” filter). However, the future will show how much these types of filters can deliver in the lab setting. Table 2 below shows further examples of extensive search of beneficial filters using parameter scanning and multiple data sets considering both mimicking and stereotype filter approach.

TABLE 2

| Dataset | | | | Z-space | Epochs of | | Size | | | R change | | Err. change | |
|---|---|---|---|---|---|---|---|---|---|---|---|---|---|
| Filter | Classifier | Input | Filter type | dim | training | Batch size | Layer 1 | Layer 2 | Layer 3 | Pre-filter | Post-filter | Pre-filter | Post-filter |
| 6 | 6 | 9 | Mimicking | 9 | 124 | 117 | 40 | 80 | 160 | 0.05 | 0.32 | 6052 | 4720 |
| 4 | 1 | 4 | Stereotyping | 12 | 134 | 88 | 40 | 80 | 160 | 0.12 | 0.25 | 1273 | 1257 |
| 6 | 6 | 7 | Mimicking | 11 | 173 | 59 | 39 | 78 | 156 | 0.03 | 0.16 | 1454 | 1026 |
| 6 | 6 | 1 | Mimicking | 12 | 172 | 98 | 53 | 106 | 212 | 0.01 | 0.04 | 1766 | 1247 |
| 9 | 2 | 9 | Stereotyping | 11 | 138 | 111 | 35 | 70 | 140 | 0.02 | 0.04 | 2482 | 2160 |
| 6 | 6 | 7 | Mimicking | 12 | 140 | 109 | 31 | 62 | 124 | 0.02 | 0.05 | 1233 | 1124 |
| 5 | 6 | 5 | Stereotyping | 12 | 183 | 119 | 48 | 96 | 192 | 0.15 | 0.18 | 631 | 604 |
| 6 | 6 | 9 | Mimicking | 8 | 132 | 69 | 13 | 26 | 52 | 0.06 | 0.1 | 6259 | 3972 |
| 9 | 9 | 5 | Mimicking | 9 | 145 | 108 | 19 | 38 | 76 | 0.14 | 0.14 | 3506 | 3375 |
| 6 | 6 | 9 | Mimicking | 10 | 179 | 87 | 14 | 28 | 56 | 0.15 | 0.44 | 4486 | 4221 |
| 9 | 6 | 9 | Stereotyping | 10 | 184 | 111 | 20 | 40 | 80 | 0.01 | 0.04 | 3601 | 2832 |
| 3 | 1 | 3 | Stereotyping | 12 | 137 | 108 | 46 | 92 | 184 | 0.02 | 0.15 | 2736 | 2623 |
| 3 | 3 | 5 | Mimicking | 11 | 167 | 69 | 28 | 56 | 112 | 0.26 | 0.26 | 2174 | 1177 |
| 6 | 6 | 2 | Mimicking | 10 | 121 | 85 | 53 | 106 | 212 | 0.02 | 0.06 | 1685 | 1306 |
| 6 | 6 | 7 | Mimicking | 7 | 121 | 119 | 19 | 38 | 76 | 0.07 | 0.07 | 1318 | 784 |
| 7 | 7 | 9 | Mimicking | 7 | 179 | 102 | 25 | 50 | 100 | 0.08 | 0.13 | 5522 | 4896 |
| 6 | 9 | 6 | Mimicking | 11 | 180 | 58 | 26 | 52 | 104 | 0.14 | 0.19 | 4518 | 4467 |
| 9 | 9 | 6 | Mimicking | 8 | 112 | 105 | 54 | 108 | 216 | 0.08 | 0.12 | 4065 | 4050 |
| 6 | 6 | 3 | Mimicking | 11 | 176 | 76 | 38 | 76 | 152 | 0.03 | 0.07 | 2924 | 2868 |
| 6 | 6 | 4 | Mimicking | 8 | 150 | 77 | 11 | 22 | 44 | 0.08 | 0.18 | 1493 | 1062 |
| 5 | 6 | 5 | Stereotyping | 12 | 181 | 78 | 23 | 46 | 92 | 0.12 | 0.17 | 975 | 917 |
| 5 | 6 | 5 | Stereotyping | 7 | 116 | 75 | 48 | 96 | 192 | 0.07 | 0.11 | 1110 | 680 |
| 6 | 8 | 6 | Stereotyping | 11 | 156 | 81 | 17 | 34 | 68 | 0.14 | 0.17 | 2244 | 2228 |
| 5 | 1 | 5 | Stereotyping | 12 | 164 | 75 | 27 | 54 | 108 | 0.02 | 0.16 | 571 | 568 |
| 3 | 1 | 3 | Stereotyping | 11 | 111 | 78 | 18 | 36 | 72 | 0.08 | 0.12 | 2884 | 2821 |
| 1 | 1 | 8 | Mimicking | 12 | 167 | 77 | 33 | 66 | 132 | 0.16 | 0.26 | 2465 | 2230 |
| 6 | 6 | 5 | Stereotyping | 8 | 120 | 75 | 24 | 48 | 96 | 0.07 | 0.09 | 1042 | 926 |
| 9 | 5 | 9 | Stereotyping | 12 | 114 | 110 | 22 | 44 | 88 | 0.02 | 0.09 | 2404 | 2180 |
| 3 | 6 | 3 | Stereotyping | 8 | 152 | 99 | 51 | 102 | 204 | 0.08 | 0.26 | 3244 | 3243 |
| 6 | 9 | 6 | Stereotyping | 9 | 119 | 123 | 43 | 86 | 172 | 0.03 | 0.14 | 4206 | 4149 |
| 3 | 3 | 9 | Mimicking | 8 | 156 | 120 | 25 | 50 | 100 | 0.15 | 0.16 | 1532 | 439 |
| 5 | 7 | 5 | Stereotyping | 10 | 159 | 111 | 35 | 70 | 140 | 0.12 | 0.15 | 1030 | 834 |
| 6 | 8 | 6 | Stereotyping | 9 | 113 | 120 | 15 | 30 | 60 | 0.05 | 0.07 | 2027 | 1757 |
| 6 | 6 | 4 | Mimicking | 9 | 135 | 96 | 38 | 76 | 152 | 0.04 | 0.08 | 1303 | 1052 |
| 6 | 6 | 4 | Mimicking | 8 | 136 | 66 | 9 | 18 | 36 | 0.02 | 0.18 | 1437 | 1052 |
| 4 | 1 | 4 | Stereotyping | 10 | 161 | 94 | 38 | 76 | 152 | 0.17 | 0.18 | 1780 | 1772 |
| 8 | 6 | 8 | Stereotyping | 9 | 123 | 93 | 45 | 90 | 180 | 0.13 | 0.14 | 2010 | 1934 |
| 6 | 6 | 5 | Mimicking | 11 | 125 | 67 | 43 | 86 | 172 | 0.15 | 0.22 | 1517 | 547 |
| 3 | 3 | 9 | Mimicking | 12 | 122 | 115 | 33 | 66 | 132 | 0.04 | 0.32 | 945 | 554 |

Here, it has been shown that a points-based HealthIndex could be incrementally built up by adding up positive and negative health points taken from predictions made on non-native data. The development of a broader HealthIndex is based on the growing number of public data sets. In particular, the bottleneck problem of how non-native predictions was addressed on how it could be made on data that could come from a different tissue, or data generation platform.

The concept of making age predictions using regression or machine learning methods, comparing the predictions (mAge) with chronological age, and attributing the discrepancy to differential health and aging is a growing endeavor. More general health predictions based on the wide variety of health annotations found in public methylation data sets could be an important extension to this activity. Each data set may frequently only be annotated with a single binary health annotation but can be used to create an (arguably) generally applicable predictor. Applied en masse, such predictions, while they may not have a specific or sufficiently reliable diagnostic value in isolation, can be argued to give a broad picture of health, that could be grouped by clinical categories.

Here, it has been addressed that some issues pertaining to making component predictions on non-native data. In particular, non-native predictions can be made both robust and diverse as described herein. This is important, as non-native predictions are frequently plagued either by being highly uniform or not repeatable. Furthermore, unsupervised Variational Auto Encoders (VAEs) was used to visualize and confirm the supervised predictions in a transform space.

In addition, by optimizing VAE settings and predictions by trial and error, the method described herein is able to present novel parameter selections, enhance the predictions on the native (intra-data set) data, and then apply the optimized “filter” on non-native data to enhance the predictions and ultimately enable the HealthIndex idea (see Table 3 below). Table 3 shows a preliminary HealthIndex, including the age of the subject (test data from GSE40279) and number of positive supervised prediction outcomes from 10 re-trainings for 7 different health outcomes (schizophrenia (GSE27097), smoking (GSE50660), lupus (test data from GSE59250), rheumatism (GSE42861), obesity (GSE73103), asthma (GSE40736), and alzheimer's (GSE53740)), as well as the average. While these “average health” values may not show a strong correlation with the chronological age, they are robust, diverse, available to VAE visualization and resistant to VAE filtering.

TABLE 3

| age: | SCH | SMK | LUP | RHE | OBE | AST | ALZ | avg: |
|---|---|---|---|---|---|---|---|---|
| 62 | 0 | 3 | 3 | 3 | 8 | 5 | 1 | 3 |
| 79 | 0 | 1 | 0 | 10 | 2 | 4 | 1 | 2 |
| 84 | 0 | 10 | 9 | 9 | 9 | 5 | 2 | 6 |
| 87 | 0 | 0 | 1 | 10 | 8 | 5 | 2 | 3 |
| 53 | 0 | 4 | 4 | 4 | 9 | 5 | 2 | 4 |
| 67 | 0 | 9 | 0 | 2 | 8 | 5 | 4 | 4 |
| 79 | 0 | 1 | 0 | 9 | 10 | 5 | 1 | 3 |
| 85 | 0 | 0 | 0 | 10 | 1 | 5 | 3 | 2 |
| 73 | 0 | 8 | 2 | 2 | 0 | 5 | 3 | 2 |
| 63 | 0 | 4 | 0 | 10 | 8 | 5 | 2 | 4 |
| 65 | 0 | 8 | 3 | 10 | 8 | 5 | 1 | 5 |
| 85 | 0 | 9 | 0 | 9 | 0 | 5 | 2 | 3 |
| 71 | 5 | 3 | 10 | 10 | 6 | 5 | 2 | 5 |
| 76 | 1 | 8 | 7 | 10 | 6 | 4 | 1 | 5 |
| 70 | 0 | 10 | 0 | 1 | 7 | 4 | 1 | 3 |
| 65 | 0 | 9 | 0 | 10 | 0 | 5 | 2 | 3 |
| 51 | 7 | 6 | 0 | 8 | 0 | 5 | 2 | 4 |
| 54 | 0 | 8 | 2 | 10 | 10 | 5 | 0 | 5 |
| 50 | 0 | 8 | 0 | 10 | 1 | 5 | 3 | 3 |
| 86 | 2 | 9 | 4 | 10 | 5 | 5 | 2 | 5 |
| 70 | 0 | 0 | 8 | 9 | 10 | 5 | 4 | 5 |
| 54 | 2 | 7 | 0 | 10 | 5 | 5 | 4 | 4 |
| 58 | 0 | 2 | 2 | 5 | 1 | 5 | 1 | 2 |
| 64 | 0 | 7 | 8 | 1 | 10 | 5 | 1 | 4 |
| 73 | 0 | 9 | 0 | 10 | 4 | 5 | 2 | 4 |
| 61 | 7 | 6 | 0 | 10 | 0 | 4 | 1 | 4 |
| 47 | 0 | 5 | 0 | 4 | 10 | 4 | 2 | 3 |
| 54 | 0 | 10 | 1 | 0 | 0 | 4 | 8 | 3 |
| 80 | 1 | 8 | 7 | 10 | 8 | 5 | 2 | 5 |
| 70 | 0 | 9 | 0 | 1 | 8 | 4 | 1 | 3 |
| 70 | 1 | 10 | 0 | 2 | 8 | 5 | 1 | 3 |
| 77 | 0 | 7 | 0 | 9 | 0 | 5 | 2 | 3 |
| 85 | 3 | 10 | 4 | 1 | 0 | 4 | 1 | 3 |
| 76 | 0 | 5 | 0 | 3 | 6 | 5 | 2 | 3 |
| 71 | 1 | 0 | 1 | 1 | 9 | 4 | 0 | 2 |
| 74 | 1 | 3 | 0 | 10 | 10 | 4 | 3 | 4 |
| 71 | 0 | 10 | 0 | 3 | 5 | 5 | 1 | 3 |
| 70 | 0 | 10 | 0 | 9 | 4 | 5 | 1 | 4 |
| 73 | 0 | 9 | 0 | 4 | 3 | 5 | 1 | 3 |
| 83 | 0 | 7 | 0 | 0 | 9 | 5 | 4 | 3 |
| 64 | 9 | 10 | 0 | 9 | 0 | 5 | 1 | 4 |
| 44 | 5 | 9 | 0 | 0 | 4 | 5 | 1 | 3 |
| 52 | 0 | 2 | 0 | 9 | 4 | 5 | 1 | 3 |
| 48 | 0 | 2 | 0 | 2 | 2 | 5 | 1 | 1 |
| 46 | 0 | 10 | 0 | 3 | 10 | 5 | 3 | 4 |
| 53 | 0 | 2 | 0 | 10 | 1 | 5 | 1 | 2 |
| 42 | 0 | 10 | 0 | 0 | 10 | 5 | 1 | 3 |
| 58 | 0 | 10 | 0 | 0 | 6 | 5 | 2 | 3 |
| 55 | 0 | 10 | 2 | 0 | 10 | 5 | 1 | 4 |
| 50 | 0 | 9 | 0 | 0 | 8 | 5 | 1 | 3 |
| 44 | 0 | 10 | 3 | 7 | 8 | 5 | 1 | 4 |
| 65 | 0 | 2 | 0 | 10 | 0 | 5 | 3 | 2 |
| 51 | 1 | 10 | 0 | 0 | 10 | 5 | 1 | 3 |
| 59 | 0 | 10 | 0 | 0 | 10 | 5 | 1 | 3 |
| 57 | 0 | 10 | 2 | 0 | 9 | 4 | 1 | 3 |
| 42 | 0 | 10 | 0 | 1 | 0 | 5 | 0 | 2 |
| 65 | 0 | 10 | 1 | 0 | 10 | 4 | 0 | 3 |
| 58 | 0 | 10 | 0 | 1 | 10 | 5 | 1 | 3 |
| 60 | 0 | 10 | 0 | 1 | 7 | 5 | 2 | 3 |
| 66 | 1 | 10 | 0 | 3 | 8 | 5 | 2 | 4 |
| 55 | 0 | 10 | 0 | 0 | 10 | 5 | 1 | 3 |
| 38 | 0 | 10 | 0 | 0 | 9 | 5 | 0 | 3 |
| 56 | 0 | 9 | 0 | 10 | 10 | 4 | 1 | 4 |
| 55 | 0 | 8 | 0 | 2 | 2 | 4 | 1 | 2 |
| 43 | 0 | 10 | 0 | 9 | 7 | 4 | 2 | 4 |
| 83 | 2 | 10 | 4 | 9 | 8 | 4 | 2 | 5 |
| 64 | 2 | 10 | 0 | 9 | 2 | 4 | 1 | 4 |
| 58 | 7 | 10 | 10 | 10 | 9 | 4 | 2 | 7 |
| 66 | 0 | 10 | 1 | 1 | 10 | 5 | 2 | 4 |
| 58 | 0 | 9 | 0 | 0 | 1 | 5 | 2 | 2 |
| 65 | 3 | 9 | 2 | 0 | 1 | 4 | 2 | 3 |
| 90 | 0 | 10 | 0 | 0 | 10 | 5 | 1 | 3 |
| 52 | 0 | 10 | 0 | 1 | 8 | 5 | 1 | 3 |
| 82 | 0 | 10 | 0 | 10 | 9 | 5 | 3 | 5 |
| 75 | 0 | 10 | 7 | 1 | 10 | 4 | 1 | 4 |
| 65 | 3 | 10 | 4 | 9 | 10 | 5 | 3 | 6 |
| 45 | 0 | 10 | 0 | 1 | 10 | 4 | 1 | 3 |
| 33 | 0 | 10 | 0 | 1 | 6 | 5 | 1 | 3 |
| 34 | 1 | 8 | 0 | 1 | 6 | 5 | 1 | 3 |
| 23 | 1 | 8 | 0 | 1 | 0 | 5 | 1 | 2 |
| 44 | 0 | 8 | 0 | 10 | 9 | 5 | 2 | 4 |
| 49 | 1 | 8 | 0 | 1 | 7 | 5 | 2 | 3 |
| 34 | 3 | 0 | 0 | 9 | 3 | 5 | 1 | 3 |
| 83 | 0 | 8 | 3 | 10 | 6 | 4 | 1 | 4 |
| 82 | 0 | 10 | 3 | 10 | 10 | 4 | 1 | 5 |
| 73 | 0 | 10 | 0 | 1 | 8 | 5 | 1 | 3 |
| 85 | 0 | 6 | 1 | 1 | 0 | 5 | 2 | 2 |
| 87 | 0 | 6 | 0 | 1 | 5 | 5 | 1 | 2 |
| 76 | 0 | 6 | 0 | 1 | 10 | 5 | 1 | 3 |
| 81 | 0 | 10 | 0 | 2 | 0 | 5 | 1 | 2 |
| 69 | 0 | 6 | 0 | 9 | 8 | 5 | 0 | 4 |
| 85 | 0 | 8 | 0 | 1 | 0 | 5 | 1 | 2 |
| 76 | 0 | 9 | 0 | 9 | 5 | 5 | 2 | 4 |
| 87 | 0 | 2 | 0 | 2 | 0 | 5 | 3 | 1 |
| 88 | 0 | 10 | 0 | 10 | 0 | 4 | 0 | 3 |
| 72 | 0 | 0 | 2 | 0 | 10 | 4 | 2 | 2 |
| 54 | 0 | 2 | 0 | 1 | 8 | 5 | 1 | 2 |
| 74 | 0 | 9 | 3 | 8 | 10 | 4 | 1 | 5 |
| 66 | 0 | 0 | 0 | 7 | 6 | 4 | 1 | 2 |
| 69 | 0 | 10 | 0 | 3 | 10 | 4 | 0 | 3 |

The embodiments disclosed herein are illustrative of certain underlying principles. Modifications to the disclosed embodiments including alternative embodiments may be employed, and are within the skill of persons of ordinary skill in the art and within the scope of the claims. The teachings and the claims are not limited to embodiments precisely as shown and described.

What is claimed is:

1. A method for detecting differences in DNA methylation in a subject, comprising:
obtaining a DNA sample from a subject;
processing the DNA sample;
detecting the CpG short tandem nucleic acid sequence in the DNA sample;
comparing the CpG short tandem nucleic acid sequence to a known population; and
providing results to the subject.

2. The method of claim 1, wherein the DNA sample is derived from human cells, tissues, blood, body fluids, urine, saliva, feces or a combination thereof; preferably plasma or urine free DNA.

3. The method of claim 1, wherein comparing the CpG short tandem nucleic acid sequence in the DNA sample further comprises comparing the degree of methylation in a DNA sample from a normal population.

4. The method of claim 1, further comprising identifying the abnormal state associated with differentially methylated CpG islands.

5. The method of claim 1, further comprising providing the subject one or more nutritional supplement or pharmaceutical based on the provided results.

6. A method for detecting a condition associated with aging, comprising:
obtaining a DNA sample from a subject;
detecting the methylation of at least one of the nucleic acid sequences or a combination thereof;
comparing the DNA sample to a known population; and
identifying a course of treatment to the subject based on the condition associated with aging.

7. The method of claim 6, wherein the DNA sample is derived from human cells, tissues, blood, body fluids, urine, saliva, feces or a combination thereof; preferably plasma or urine free DNA.

8. The method of claim 6, further comprising comparing a CpG short tandem nucleic acid sequence in the DNA sample by comparing the degree of methylation in a DNA sample from the known population.

9. The method of claim 6, further comprising identifying the abnormal state associated with differentially methylated CpG islands.

10. The method of claim 6, wherein the course of treatment comprises providing one or more nutritional supplement or pharmaceutical based on condition associated with aging.

11. A method for treating a condition associated with aging, comprising:
obtaining a DNA sample from a subject;
detecting the methylation of at least one of the nucleic acid sequences or a combination thereof;
comparing the DNA sample to a known population; and
providing a course of treatment to the subject based on the condition associated with aging.

12. The method of claim 11, wherein the DNA sample is derived from human cells, tissues, blood, body fluids, urine, saliva, feces or a combination thereof; preferably plasma or urine free DNA.

13. The method of claim 11, further comprising comparing a CpG short tandem nucleic acid sequence in the DNA sample by comparing the degree of methylation in a DNA sample from the known population.

14. The method of claim 11, further comprising identifying the abnormal state associated with differentially methylated CpG islands.

15. The method of claim 11, wherein the course of treatment comprises providing one or more nutritional supplement or pharmaceutical based on condition associated with aging.
